SEQUENCE LISTING (15 sequences)

SEQ ID NO: 1 - DNA, 20 bp, Artificial sequence
  CAS9 targeting site
gcgaggcgttggataagcct

SEQ ID NO: 2 - DNA, 21 bp, Artificial sequence
  TK forward primer
gtacccgagccgatgacttac

SEQ ID NO: 3 - DNA, 16 bp, Artificial sequence
  TK reverse primer
cccggccgatatctca

SEQ ID NO: 4 - DNA, 29 bp, Artificial sequence
  TK probe
cttccgagacaatcgcgaacatctacacc

SEQ ID NO: 5 - DNA, 15 bp, Artificial sequence
  RPP30 forward primer
gatttggacctgcga

SEQ ID NO: 6 - DNA, 15 bp, Artificial sequence
  RPP30 reverse primer
gcggctgtctccaca

SEQ ID NO: 7 - DNA, 16 bp, Artificial sequence
  RPP30 probe
ctgacctgaaggctct

SEQ ID NO: 8 - DNA, 20 bp, Artificial sequence
  R1 primer
ggctggacagagaacaggac

SEQ ID NO: 9 - DNA, 19 bp, Artificial sequence
  F2 primer
gcccccgaaccttcaaaga

SEQ ID NO: 10 - DNA, 18 bp, Artificial sequence
  R2 primer
ctgcacgccgtaggtcag

SEQ ID NO: 11 - DNA, 20 bp, Artificial sequence
  F3 primer
ggagatgggggaggctaact

SEQ ID NO: 12 - DNA, 12 bp, Artificial sequence
  Figure 1f - sequence 1
tacgtcggaagc

SEQ ID NO: 13 - DNA, 12 bp, Artificial sequence
  Figure 1f - sequence 2
ggacctatggtg

SEQ ID NO: 14 - DNA, 15 bp, Artificial sequence
  Figure 1f - sequence 3
tacaagtgagggcgc

SEQ ID NO: 15 - DNA, 16 bp, Artificial sequence
  Figure 1f - sequence 4
cgttggttaagcctgg

(19) United States
(12) Patent Application Publication
(10) Pub. No.: US 2021/0247385 A1
(43) Pub. Date: Aug. 12, 2021

(54) METHODS FOR ASSESSING TRANSENDOTHELIAL BARRIER INTEGRITY

(71) Applicant: Hoffmann-La Roche Inc., Little Falls, NJ (US)

(72) Inventors: Chad A. COWAN, Boston, MA (US); Claas Aiko MEYER, Basel (CH); Filip ROUDNICKY, Basel (CH); Jitao David ZHANG, Basel (CH)

(73) Assignee: Hoffmann-La Roche Inc., Little Falls, NJ (US)

(21) Appl. No.: 17/180,406

(22) Filed: Feb. 19, 2021

Related U.S. Application Data
(63) Continuation of application No. PCT/EP2019/072070, filed on Aug. 19, 2019.

(30) Foreign Application Priority Data
Aug. 21, 2018 (EP) 18190039.0

(51) Int. Cl. G01N 33/50; C12Q 1/6897; C12N 5/071; A61K 31/4439

(52) U.S. Cl. CPC G01N 33/5064; C12Q 1/6897; G01N 33/5032; G01N 33/5023; C12N 2506/03; A61K 31/4439; C12N 2503/02; C12N 2510/00; C12N 5/069

(57) ABSTRACT

This application relates to a method for identifying a drug candidate capable of increasing or decreasing barrier tissue integrity of endothelial cells. Moreover, this application relates to the use of a tight junction gene transcriptional reporter as a surrogate marker of transendothelial barrier integrity.

### CROSS-REFERENCE TO RELATED APPLICATIONS

This application is a continuation of International Application No. PCT/EP2019/072070, filed Aug. 19, 2019, which claims priority to EP Application No. 18190039.0, filed Aug. 21, 2018, the disclosures of which are incorporated herein by reference in their entireties.

### SEQUENCE LISTING

This application contains a Sequence Listing which has been submitted electronically in ASCII format and is hereby incorporated by reference in its entirety. Said ASCII copy, created on Feb. 11, 2021, is named “P34956-US_Sequence_Listing_ST25.txt” and is 4,096 bytes in size.

### FIELD OF THE INVENTION

This application relates to a method for identifying a drug candidate capable of increasing or decreasing barrier integrity of endothelial cells. Moreover, this application relates to the use of a tight junction gene transcriptional reporter as a surrogate marker of transendothelial barrier integrity.

### BACKGROUND

Endothelial cell barrier that forms blood-retinal (BRB) and blood-brain barrier (BBB) is critical for homeostasis and preventing toxicity and infection to eye and brain (Engelhardt B, Liebner S. Cell and tissue research. 2014; 355(3):687-99, Diaz-Coranguez M, Ramos C, Antonetti D A. Vision research. 2017; 139:123-37). Disruption of the endothelial cell barrier is implicated in several disease of retina, for example familial exudative vitreoretinopathy (Gilmour D F. Eye (London, England). 2015; 29(1):1-14) age-related macular degeneration and diabetic retinopathy (Klaassen I, Van Noorden C J, Schlingemann R O. Progress in retinal and eye research. 2013; 34:19-48), and neurological disease of the brain (Zhao Z, Nelson A R, Betsholtz C, Zlokovic B V. Cell. 2015; 163(5):1064-78). Endothelial cells (ECs) express specialized set of tight junctions and transporters (Luissint A C, Artus C, Glacial F, Ganeshamoorthy K, Couraud P O. Fluids and barriers of the CNS. 2012; 9(1):23) that forms a selective barrier of high resistance.

Isolated primary ECs from BRB and BBB in vitro quickly lose their barrier properties found in vivo, therefore there were several studies that used complicated co-cultured systems in 2-D and 3-D with primary cells from neurovascular junction that mimicked the in vivo conditions (Helms H C, Abbott N J, Burek M, Cecchelli R, Couraud P O, Deli M A, et al. Journal of cerebral blood flow and metabolism. 2016; 36(5):862-90, Nzou G, Wicks R T, Wicks E E, Seale S A, Sane C H, Chen A, et al. Scientific reports. 2018; 8(1):7413). The main disadvantage of primary cells for drug discovery is their limited lifespan and availability (Eglen R, Reisine T. 2011; 9(2):108-24). Pluripotent-stem cells have the potential to differentiate into any type of adult cell type (Zhu Z, Huangfu D. Development (Cambridge, England). 2013; 140(4):705-17) and they have been used for modeling the blood-brain barrier (Lippmann E S, Azarin S M, Kay J E, Nessler R A, Wilson H K, Al-Ahmad A, et al. Nature biotechnology. 2012; 30(8):783-91, Canfield S G, Stebbins M J, Morales B S, Asai S W, Vatine G D, Svendsen C N, et al. Journal of neurochemistry. 2017; 140(6):874-88). Main disadvantages of these published models are that they are highly sophisticated and difficult to accurately reproduce, making them difficult to adapt for drug discovery.

Thus there remains a need for robust and meaningful models of transendothelial barrier integrity (TBI) and respective cell culture methods suitable to generate large quantities of ECs capable of establishing high resistance in vitro TBI as a model to study BRB and BBB in healthy and diseased conditions.

The present inventors have previously established a simple and scalable 6-day protocol to differentiate human pluripotent stem cells into functional endothelial cells (Patsch C, Challet-Meylan L, Thoma E C, Urich E, Heckel T, O'Sullivan J F, et al. Nature cell biology. 2015; 17(8):994-1003).

Here, the inventors generated an in vitro model of endothelial cells of high TBI that can be used to find novel pathways and targets for treatment of diseases with endothelial cells disruption, in particular in a drug screening and/or development setting.

### SUMMARY OF THE INVENTION

Provided herein is an in vitro method for identifying a drug candidate capable of i) increasing in vivo transendothelial barrier integrity (TBI) or ii) decreasing in vivo TBI of endothelial cells (ECs) comprising the steps of:
a) providing ECs comprising a reporter gene under the control of a tight junction gene promoter, wherein the ECs are enriched for cells expressing the reporter gene;
b) contacting the ECs with the drug candidate;
c) measuring in vitro TBI before and after contacting the ECs with the drug candidate, or measuring in vitro TBI of the ECs contacted with the drug candidate and in parallel measuring in vitro TBI of ECs not contacted with the drug candidate;
   wherein (i) a higher in vitro TBI of the ECs contacted with the drug candidate compared with the in vitro TBI of the ECs not contacted with the drug candidate is indicative of a drug capable of increasing in vivo TBI of ECs, and (ii) a lower in vitro TBI of the ECs contacted with the drug candidate compared with the in vitro TBI of the ECs not contacted with the drug candidate is indicative of a drug capable of decreasing in vivo TBI of ECs.

In one embodiment, step c) comprises measuring the transendothelial electrical resistance (TEER) wherein the measured TEER is indicative for in vitro TBI.

In one embodiment, step c) comprises measuring the expression of the reporter gene wherein the expression of the reporter gene is indicative for in vitro TBI.

In one embodiment, the tight junction gene is selected from the group consisting of CLDN5, ocludin (OCLN) and MARVELD3, in particular wherein the tight junction gene is CLDN5.

In one embodiment, the ECs are differentiated from pluripotent stem cells, in particular wherein the pluripotent stem cells are human cells.

In one embodiment, the pluripotent stem cells are derived from a subject suffering from a disease associated with vascular complications.

In one embodiment, a polynucleotide encoding the reporter gene is inserted at the 3′ end of the tight junction gene, in particular wherein (i) a tight junction gene reporter gene fusion protein is expressed or (ii) the reporter gene is expressed from an internal ribosomal entry site (IRES), or (iii) a tight junction gene reporter gene fusion protein is expressed and subsequently processed to individual tight junction protein and reporter protein.

In one embodiment, a polynucleotide encoding a self-cleaving peptide is introduced between the tight junction gene and the reporter gene, in particular wherein the self-cleaving peptide is the P2A self-cleaving peptide.

In one embodiment, activation of the promoter of the tight junction gene leads to expression of the reporter gene.

In one embodiment, the cells are enriched for cells expressing the reporter gene in step a) by fluorescence activated cell sorting (FACS) or magnetic activated cell sorting (MACS).

In one embodiment, the method as herein provided is performed in a high-throughput format.

In one embodiment, the method as herein provided is used to screen molecules in a drug development setting, in particular for high-throughput screening a drug candidate compound library.

In one embodiment, provided is a cell culture produced according to step 1) a) of the method as described herein, wherein the fraction of cells expressing the tight junction gene is higher than 30%, 40%, 50%, 60%, 70%, 80%, 90% or 95%.

In one embodiment, provided is a cell capable of expressing a reporter gene, wherein expression of the reporter gene is under the control of the promoter of a tight junction gene, is selected from the group consisting of CLDN5, ocludin (OCLN) and MARVELD3.

In one embodiment, provided is 2-[3-(6-Methyl-2-pyridinyl)-1H-pyrazol-4-yl]-1,5-naphthyridine or 4-[4-[3-(2-Pyridinyl)-1H-pyrazol-4-yl]-2-pyridinyl]-N-(tetrahydro-2H-pyran-4-yl)-benzamide for use in the treatment of a disease associated with vascular complications.

### SHORT DESCRIPTION OF THE FIGURES

The patent or application file contains at least one drawing executed in color. Copies of this patent or patent application publication with color drawing(s) will be provided by the Office upon request and payment of the necessary fee.

FIG. 1A-FIG. 1F: Genome editing of the CLDN5 transcriptional reporter. Schematic of the targeting strategy for generating CLDN5-P2A-GFP reporter. SgRNA was designed in the vicinity of the stop codon of CLDN5 while a donor vector was generated to carry a promoterless P2A-GFP sequence flanked by two homology arms (HAs) at each end with piggyBac inverted terminal repeats (ITR). (LHA-left homology arm, RHA-right homology arm, PURO-puromycin, tTK-truncated thymidine kinase). Targeting was performed in two steps, first the double stranded break caused by Cas9 and sgRNA was repaired by homologous recombination between CLDN5 and donor template and subsequently the resistance cassette was removed by excision-only piggybac transposase (FIG. 1A). Schematic map of donor vector (FIG. 1B). Detection of successful integration of reporter by PCR and gel electrophoresis after genome editing and puromycin selection (cell pool-genome editing-puromycin selected (CPGP)) (FIG. 1C). Detection of successful excision of resistance cassette by PCR and gel electrophoresis (cell pool-excision (CPE)) (FIG. 1D). Validation of clones by PCR and gel electrophoresis (FIG. 1E). Sanger sequence of CLDN5 locus of the positive clones (FIG. 1F). FIG. 1F discloses SEQ ID NOS 12-15, respectively, in order of appearance.

FIG. 2A-FIG. 2F: Generation and characterization of Stem-cell derived endothelial cells comprising a CLDN5 reporter. Human pluripotent stem-cell differentiation of WT and CLDN5-GFP reporter lines into endothelial cells with Fluorescence-activated cell sorting of ECs from CLDN5-GFP ECs (FIG. 2A). Electric cell-substrate impedance sensing of GFP+ and GFP− sorted cells observed in real time (FIG. 2B). Representation values of one clone, Spearman correlation of significantly up- or down-regulated proteins and their respective mRNAs measured by mass spectrometry and RNA-seq (FIG. 2C). Relative RNA and protein expression for CLDN5 (FIG. 2D), for OCLN, MARVELD3 and PECAM1 (FIG. 2E) and for VEGFA receptor 2 (KDR) (FIG. 2F). Columns show means±SD. **=p<0.01, ***=p<0.001

FIG. 3A-FIG. 3E: CLDN5-GFP+ ECs show functional response of high endothelial cell barrier. GFP+ cells were stimulated with 50 ng/mL VEGFA and the electric cell-substrate impedance was measured in real time (FIG. 3A). After 2 days of VEGFA treatment relative GFP+ % of cells was measured with FACS (FIG. 3B). Cells were treated with 5 μM SU11248 for 2 days and the percentage of GFP+ cells was determined (FIG. 3C), impedance was measured in real time (FIG. 3D) and FITC-dextran permeability was measured (FIG. 3E). Columns show means±SD. ***=p<0.001

FIG. 4: Identification of compounds inducing EC barrier resistance. A compound library was tested in duplicate plates. Compounds were used at 5 μM and the percentage of GFP+ cells was determined 2 days post-treatment (FIG. 4). With 2-fold mean induction of percentage of GFP+ cells over DMSO, 62 compounds were identified that mapped to several target classes (e.g., TGFBR inhibitors).

FIG. 5: Rescue of transendothelial barrier integrity (TBI). Impedance real time measurement upon candidate compound co-treatment with VEGFA. GFP+ cells were incubated with 50 ng/mL VEGFA and the electric cell-substrate impedance was measured in real time (FIG. 5). Repsox (10 μM) rescues the loss-of TBI induced by VEGFA treatment. Columns show means±SD.

### DETAILED DESCRIPTION

As used herein, the term “defined medium” or “chemically defined medium” refers to a cell culture medium in which all individual constituents and their respective concentrations are known. Defined media may contain recombinant and chemically defined constituents.

As used herein the term “differentiating”, “differentiation” and “differentiate” refers to one or more steps to convert a less-differentiated cell into a somatic cell, for example to convert a pluripotent stem cell into an EC. Differentiation of a pluripotent stem cell to a EC is achieved by method described herein.

As used herein, “endothelial cells”, abbreviated “ECs”, are cells that express the specific surface marker CD144 (Cluster of Differentiation 144, also known as Cadherin 5, type 2 or vascular endothelial (VE)-cadherin, official symbol CDH5) and possess characteristics of endothelial cells, namely capillary-like tube formation, and the expression of one or more further surface markers selected from the group of, CD31 (Cluster of Differentiation 31, official symbol PECAM1), vWF (Von Willebrand factor, official symbol VWF), CD34 (Cluster of Differentiation 34, official symbol CD34), CD105 (Cluster of Differentiation 105, official symbol ENG), CD146 (Cluster of Differentiation 34, official symbol MCAM), and VEGFR-2 (kinase insert domain receptor (a type III receptor tyrosine kinase), official symbol KDR).

“Expansion medium” as used herein refers to any chemically defined medium useful for the expansion and passaging endothelial cells on a monolayer.

By “fused” is meant that the components (e.g., a tight junction gene and a reporter gene) are linked by peptide bonds, either directly or via one or more peptide linkers.

As used herein, the term “GW788388” refers to 4-[4-[3-(2-Pyridinyl)-1H-pyrazol-4-yl]-2-pyridinyl]-N-(tetrahydro-2H-pyran-4-yl)-benzamide.

As used herein, the term “growth factor” means a biologically active polypeptide or a small molecule compound which causes cell proliferation, and includes both growth factors and their analogs.

“High-throughput screening” as used herein shall be understood to signify that a large number of different disease model conditions and/or chemical compounds can be analyzed and compared, parallel and/or sequential, with the novel assay described herein. Typical, such high-throughput screening is performed in multi-well microtiter plates, e.g., in a 96 well plate or a 384 well plate or plates with 1536 or 3456 wells.

“Induction medium” as used herein refers to any chemically defined medium useful for the induction of primed cells into CD144 positive (CD144+) endothelial cells on a monolayer.

A “monolayer of pluripotent cells” as used herein means that the pluripotent stem cells are provided in single cells which are attached to the adhesive substrate in one single film, as opposed to culturing cell clumps or embryoid bodies in which a solid mass of cells in multiple layers form various three dimensional formations attached to the adhesive substrate.

“Pluripotency medium” as used herein refers to any chemically defined medium useful for the attachment of pluripotent stem cells as single cells on a monolayer while maintaining their pluripotency. Useful pluripotency media and are well known in the art also described herein. In particular embodiments as described herein, the pluripotency medium contains at least one of the following growth factors: basic fibroblast growth factor (bFGF, also depicted as Fibroblast Growth Factor 2, FGF2) and transforming growth factor β (TGFβ).

As used herein, the term “reprogramming” refers to one or more steps needed to convert a somatic cell to a less-differentiated cell, for example for converting a fibroblast cell, adipocytes, keratinocytes or leucocyte into a pluripotent stem cell. “Reprogrammed” cells refer to cells derived by reprogramming somatic cells as described herein.

As used herein, the term “Repsox” refers to 2-[3-(6-Methyl-2-pyridinyl)-1H-pyrazol-4-yl]-1,5-naphthyridine.

The term “small molecule”, or “small compound”, or “small molecule compound” as used herein, refers to organic or inorganic molecules either synthesized or found in nature, generally having a molecular weight less than 10,000 grams per mole, optionally less than 5,000 grams per mole, and optionally less than 2,000 grams per mole.

The term “somatic cell” as used herein refers to any cell forming the body of an organism that are not germline cells (e.g., sperm and ova, the cells from which they are made (gametocytes)) and undifferentiated stem cells.

The term “stem cell” as used herein refers to a cell that has the ability for self-renewal. An “undifferentiated stem cell” as used herein refers to a stem cell that has the ability to differentiate into a diverse range of cell types. As used herein, “pluripotent stem cells” refers to a stem cell that can give rise to cells of multiple cell types. Pluripotent stem cells (PSCs) include human embryonic stem cells (hESCs) and human induced pluripotent stem cells (hiPSCs). Human induced pluripotent stem cells can be derived from reprogrammed somatic cells, e.g. by transduction of four defined factors (Sox2, Oct4, Klf4, c-Myc) by methods known in the art and further described herein. Said human somatic cells can be obtained from a healthy individual or from a patient. These donor cells can be obtained from any suitable source. Preferred herein are sources that allow isolation of donor cells without invasive procedures on the human body, for example human skin cells, blood cells or cells obtainable from urine samples. Although human pluripotent stem cells are preferred, the method is also applicable to non-human pluripotent stem cells, such as primate, rodent (e.g. rat, mouse, rabbit) and dog pluripotent stem cells.

As used herein, the term “transendothelial barrier integrity”, abbreviated “TBI”, refers to a functional hallmark of endothelial cells in vitro and in vivo. Endothelial cells (ECs) act as a semi-selective barrier between the vessel lumen and surrounding tissue, controlling the passage of materials and the transit of white blood cells into and out of the bloodstream. Loss of barrier function, i.e. loss of TBI, is observed in healthy and disease conditions, e.g., would healing, vascularization as well as chronic inflammation coincide with temporary or permanent loss of TBI. TBI can be modeled in vitro by monolayers of ECs (e.g., EC cultures) produced under appropriate conditions as described herein and known in the art (e.g., short-term primary cell culture). TBI, e.g., in vitro TBI, can be measured with methods known in the art (e.g., measuring TEER and FITC-dextran permeability) and as herein described. As used herein, the term “in vitro TBI” refers to TBI of an in vitro endothelial cell culture wherein the TBI is measured across the cell monolayer in culture, e.g. between the culture vessel surface below the monolayer and the cell culture medium above the monolayer of cells (in a classical 2D cell culture setup). Accordingly, as used herein, the term “in vivo TBI” refers to the TBI of endothelial cells in vivo, wherein the TBI is established and/or determined (e.g., measured) between a vessel lumen and the surrounding tissue.

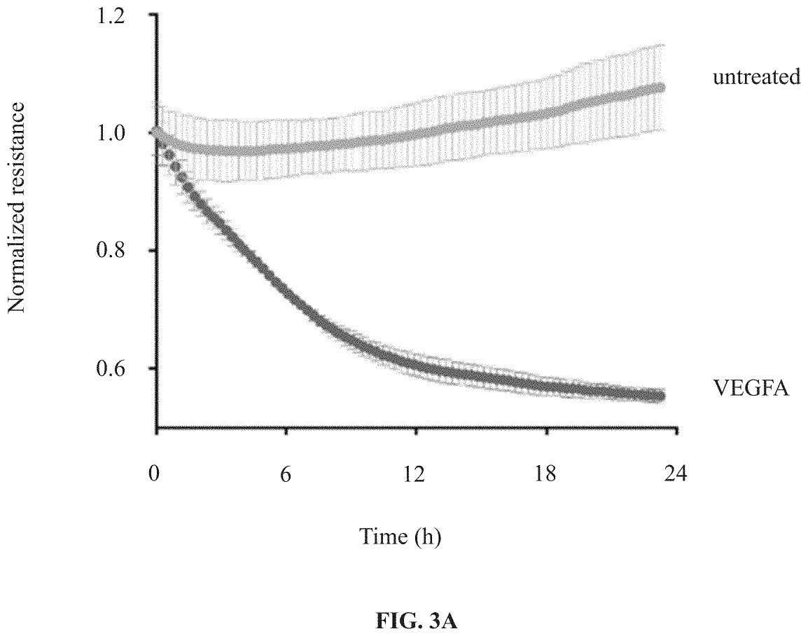

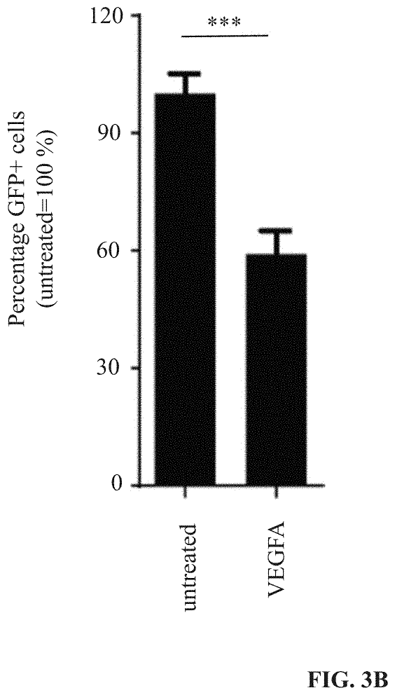

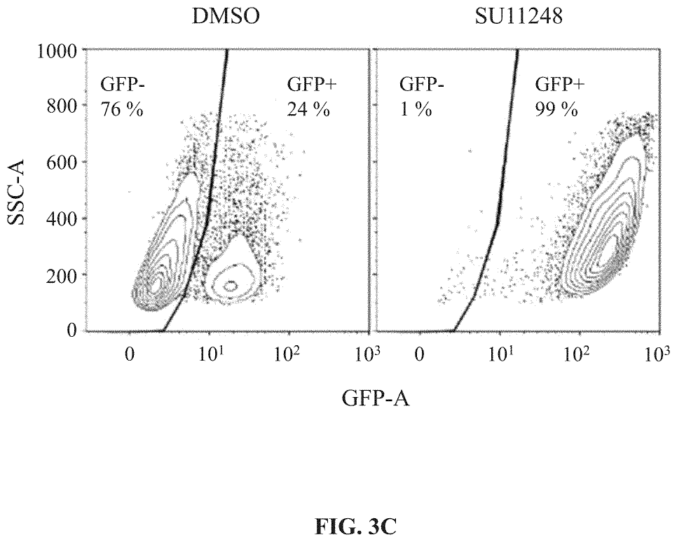

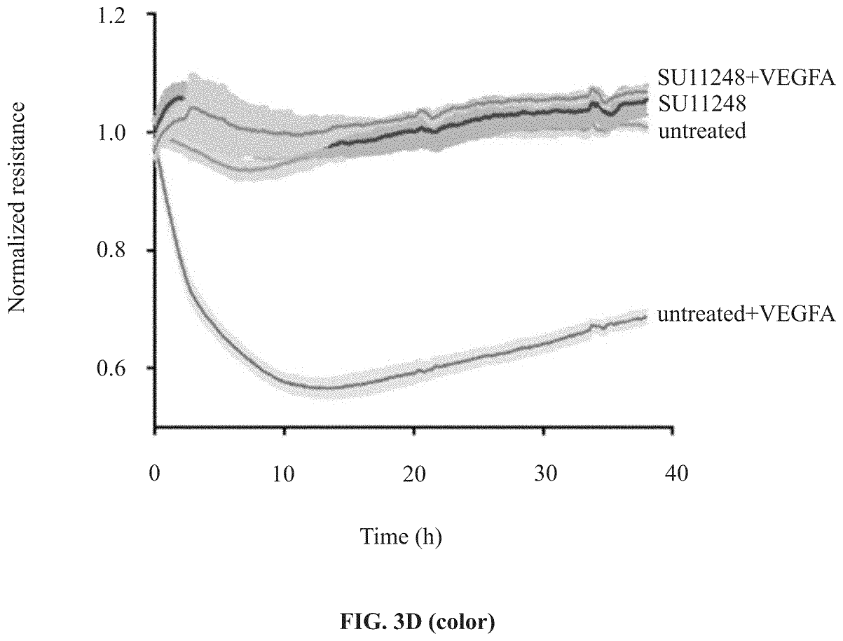

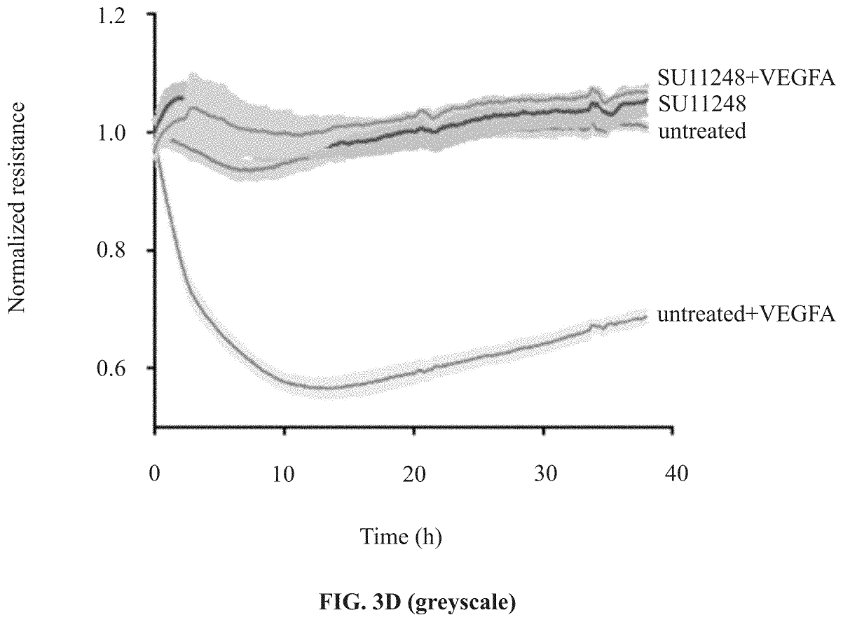

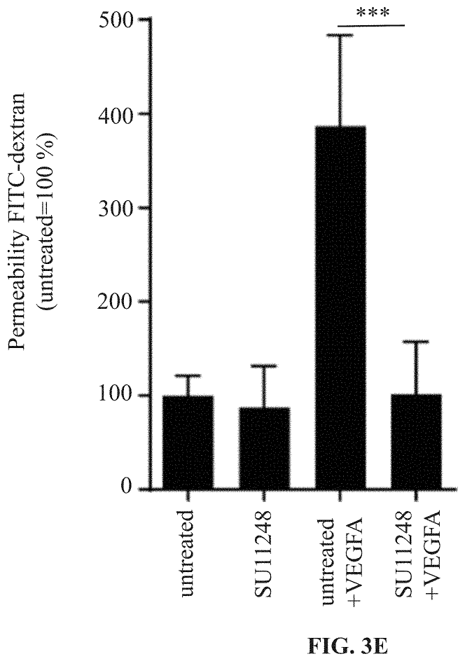

The present inventors surprisingly found that a tight junction gene transcriptional reporter can serve as a surrogate marker of TBI, i.e., the expression of the reporter gene correlates to TBI. Furthermore, the expression of the reporter gene can be used to select and enrich for cells capable of establishing high TBI in vitro. The cell cultures produced with the methods as described herein can be used to predict in vivo response to a drug candidate as herein demonstrated herein. As a proof of concept, reporter gene positive cells were treated with vascular endothelial growth factor (VEGFA), a potent vascular permeability factor in vivo, whereupon a striking loss of TBI was observed (FIG. 3A) and interestingly, a reduction of reporter gene positive cells was observed. Treatment with a broad tyrosine kinase receptor (SU11248) inhibitor led to increase of reporter gene positive cells and co-treatment of the cells with a tyrosine kinase inhibitor together with VEGFA prevented TBI breakdown (FIG. 3C). This data and further data as herein provided demonstrates that a tight junction gene transcriptional reporter as described herein can be used as a surrogate marker for EC TBI. The reporter construct as described herein, and cells comprising such reporter constructs, inter alia, are useful in methods to profile chemical libraries to find compounds that induce high endothelial barrier integrity or prevent loss of barrier breakdown.

Accordingly, provided herein is an in vitro method for identifying a drug candidate capable of i) increasing in vivo transendothelial barrier integrity (TBI) or ii) decreasing in vivo TBI of endothelial cells (ECs) comprising the steps of:
a) providing ECs comprising a reporter gene under the control of a tight junction gene promoter, in particular wherein the ECs are enriched for cells expressing the reporter gene;
b) contacting the ECs with the drug candidate;
c) measuring in vitro TBI before and after contacting the ECs with the drug candidate, or measuring in vitro TBI of the ECs contacted with the drug candidate and in parallel measuring in vitro TBI of ECs not contacted with the drug candidate;

wherein (i) a higher in vitro TBI of the ECs contacted with the drug candidate compared with the in vitro TBI of the ECs not contacted with the drug candidate is indicative of a drug capable of increasing in vivo TBI of ECs, and (ii) a lower in vitro TBI of the ECs contacted with the drug candidate compared with the in vitro TBI of the ECs not contacted with the drug candidate is indicative of a drug capable of decreasing in vivo TBI of ECs.

Without being bound to theory, the present invention provides, inter alia, cell culture models of TBI wherein in vitro TBI of ECs is assessed to establish and/or predict the effect of a drug candidate on in vivo TBI of endothelial cells. Accordingly, suitable drug candidates can be selected according to the methods as herein provided.

A TBI model with surprisingly high TBI is provided herein wherein the ECs comprise a reporter gene under the control of a tight junction gene promoter, wherein the reporter gene is operationally coupled to the activity of the tight junction gene promoter.

As used herein, a “tight junction gene promoter” refers to a gene promoter operationally coupled to a tight junction gene. Activation of the tight junction gene promoter leads to expression (transcription and translation) of the associated tight junction gene. Accordingly, operational coupling of a reporter gene with the tight junction gene promoter, e.g., by inserting DNA encoding the reporter gene into the tight junction gene locus or fusing DNA encoding the reporter gene with the DNA sequence encoding the tight junction gene, leads to expression of the reporter gene upon activation of the tight junction gene promoter. Methods for inserting a reporter into a gene locus and/or operationally coupling a reporter gene with a promoter are known in the art and also described herein.

As used herein, a “reporter gene” means a gene whose expression can be assayed. In one preferred embodiment a reporter gene is a gene that encodes a protein the production and detection of which is used as a surrogate to detect (indirectly) the activity of the tight junction promoter to be reported. Suitable reporter genes are widely known in the art and include, e.g. proteins with intrinsic fluorescence (e.g., fluorescent proteins). The expression of such proteins can be conveniently detected or monitored (e.g., in real-time) by measuring the fluorescence signal from cells (e.g., EC cultures) capable of expressing the reporter gene. Towards this end the method as described herein comprises measuring the expression level of the reporter gene wherein the expression level of the reporter gene is indicative for expression of the tight junction gene, and as such, is used as a surrogate marker for TBI. In one preferred embodiment, the expression of the reporter gene is determined by measuring fluorescence, wherein the level of fluorescence (e.g., GFP fluorescence) is indicative for TBI.

The term “protein with intrinsic fluorescence” includes wild-type fluorescent proteins and mutants that exhibit altered spectral or physical properties. The term does not include proteins that exhibit weak fluorescence by virtue only of the fluorescence contribution of non-modified tyrosine, tryptophan, histidine and phenylalanine groups within the protein. Proteins with intrinsic fluorescence are known in the art, e.g., green fluorescent protein (GFP), red fluorescent protein (RFP), Blue fluorescent protein (BFP, Heim et al. 1994, 1996), a cyan fluorescent variant known as CFP (Heim et al. 1996; Tsien 1998); a yellow fluorescent variant known as YFP (Oruro et al. 1996; Wachter et al. 1998); a violet-excitable green fluorescent variant known as Sapphire (Tsien 1998; Zapata-Hommer et al. 2003); and a cyan-excitable green fluorescing variant known as enhanced green fluorescent protein or EGFP (Yang et al. 1996). However, also enzymes whose catalytic activity can be detected are envisaged. Non-limiting examples of such enzymes are Luciferase, beta Galactosidase, Alkaline Phosphatase. Luciferase is a monomeric enzyme with a molecular weight (MW) of 61 kDa. It acts as a catalysator and is able to convert D-luciferin in the presence of Adenosine triphosphate (ATP) and Mg2+ to luciferyl adenylate. In addition, pyrophosphate (PPi) and adenosine monophosphate (AMP) are generated as byproducts. The intermediate luciferyl adenylate is then oxidized to oxyluciferin, carbon dioxide (CO2) and light. Oxyluciferin is a bioluminescent product which can be quantitatively measured in a luminometer by the light released from the reaction. Luciferase reporter assays are commercially available and known in the art, e.g., Luciferase 1000 Assay System and ONE-Glo™ Luciferase Assay System.

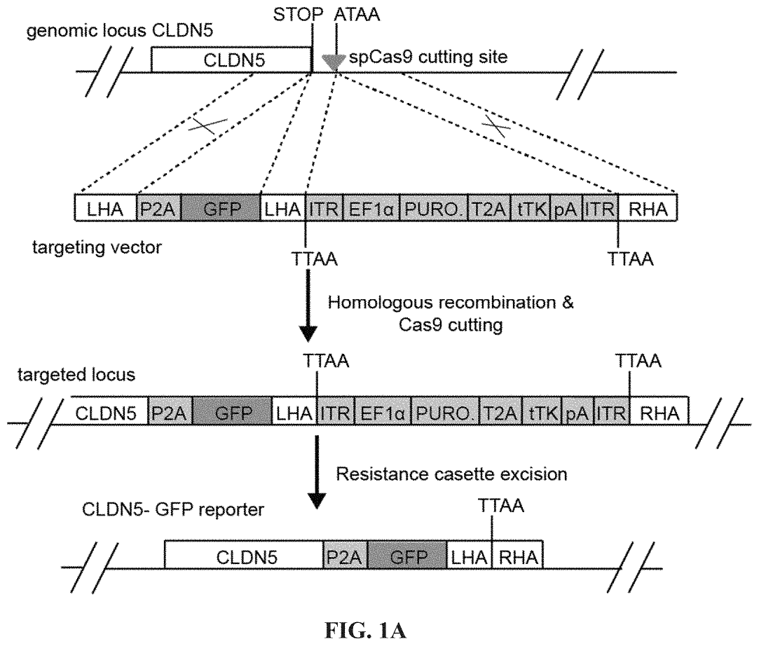

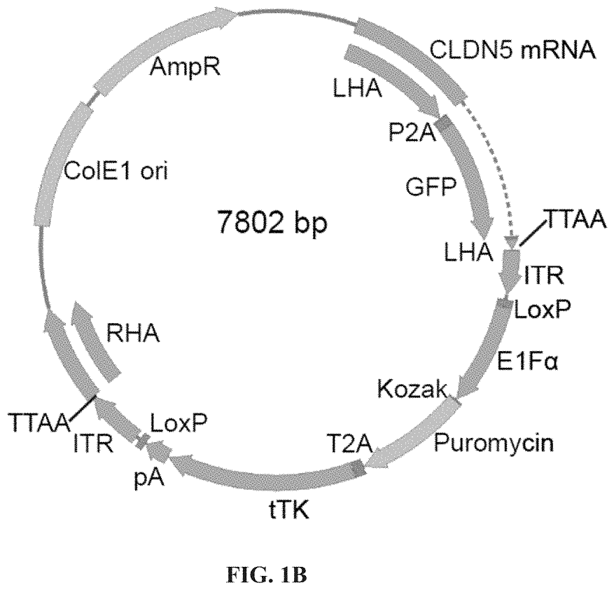

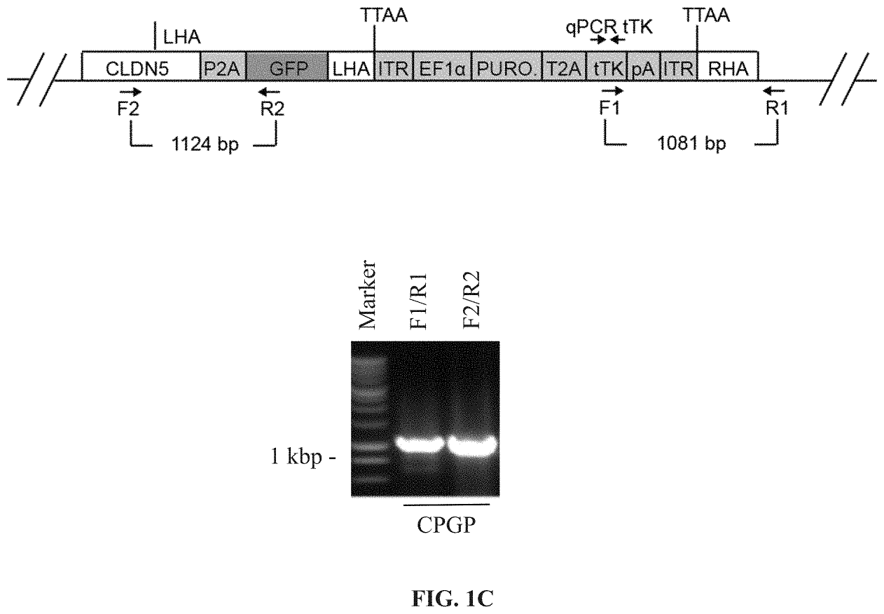

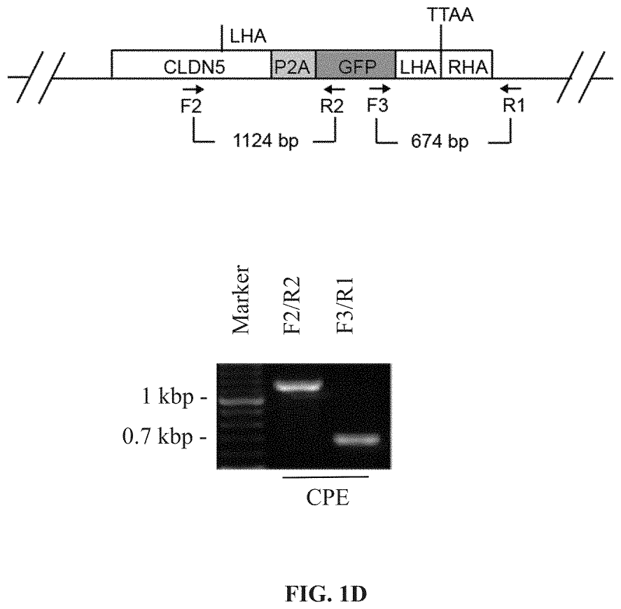

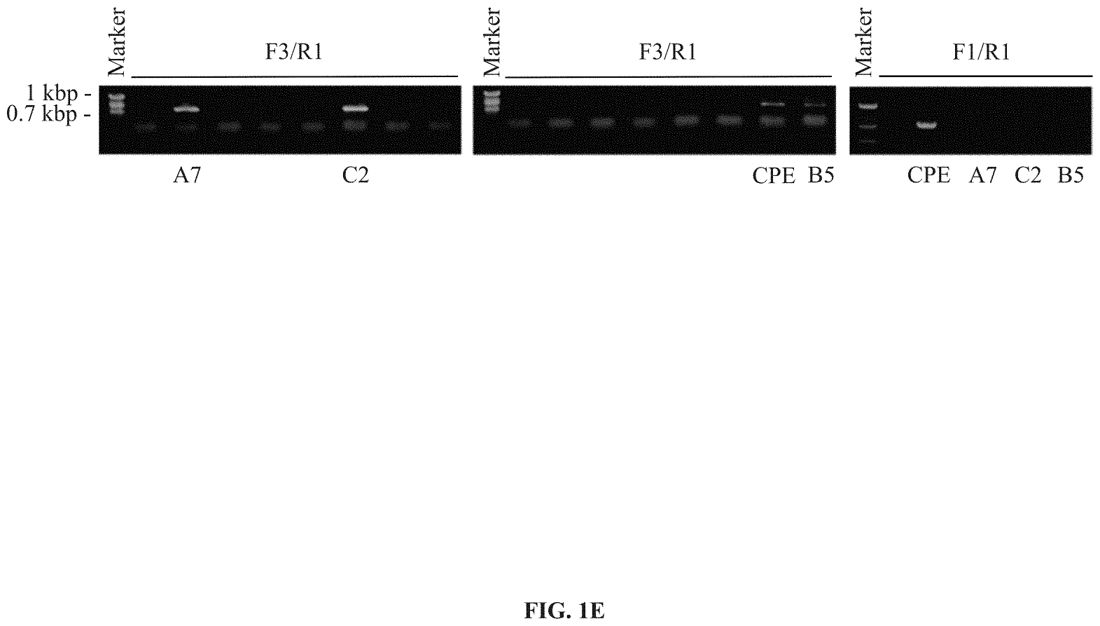

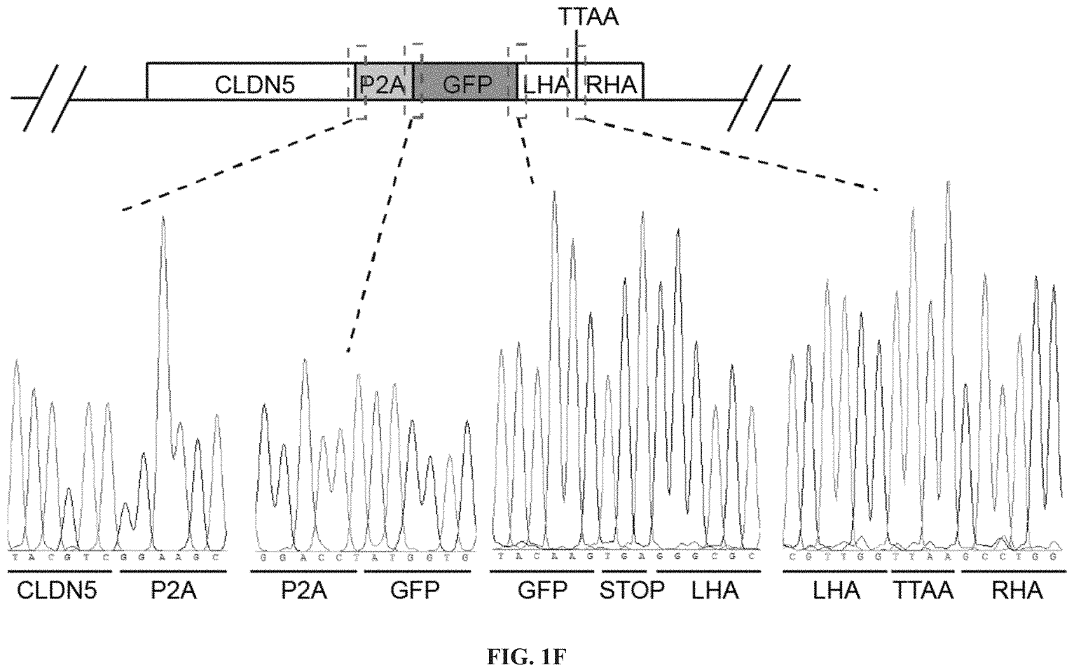

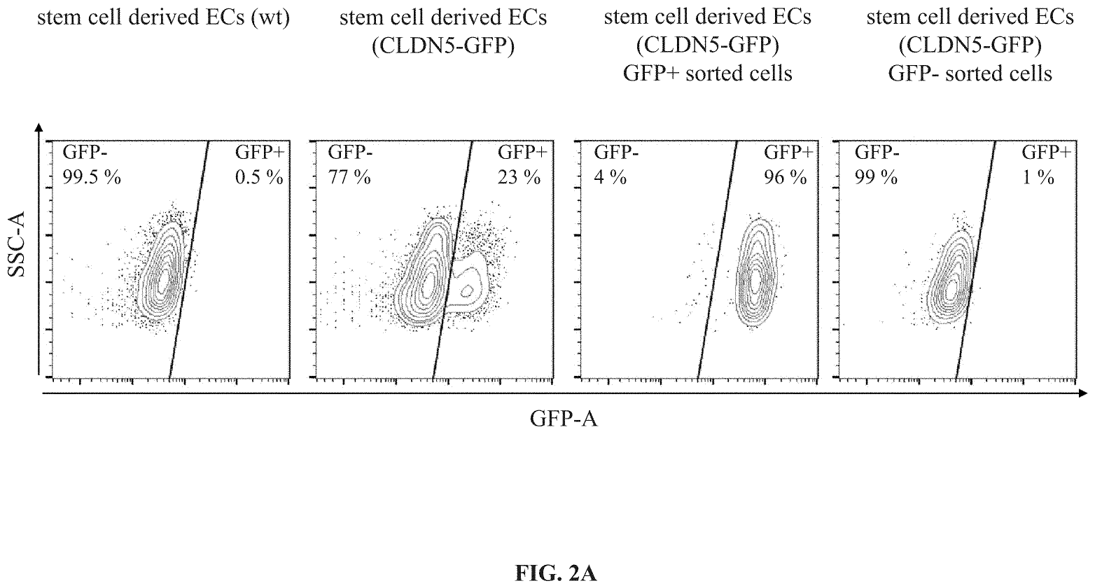

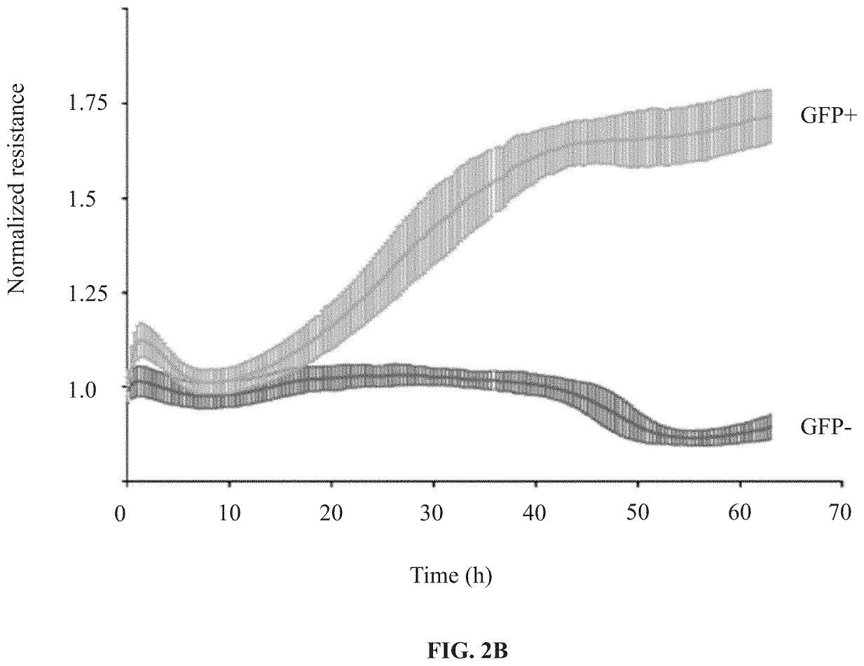

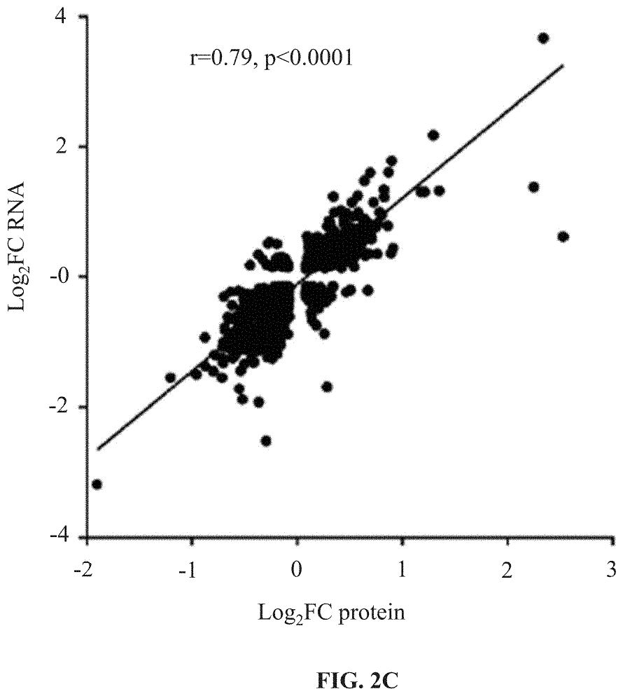

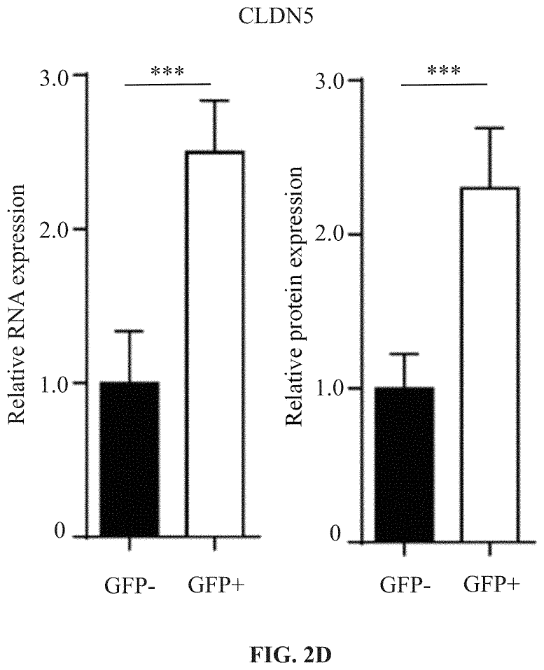

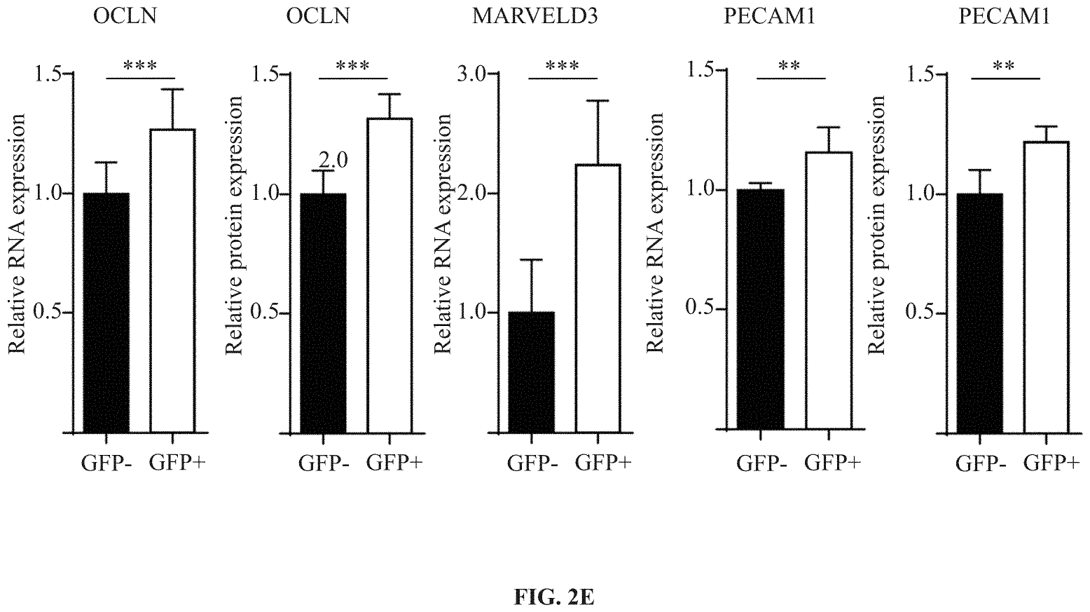

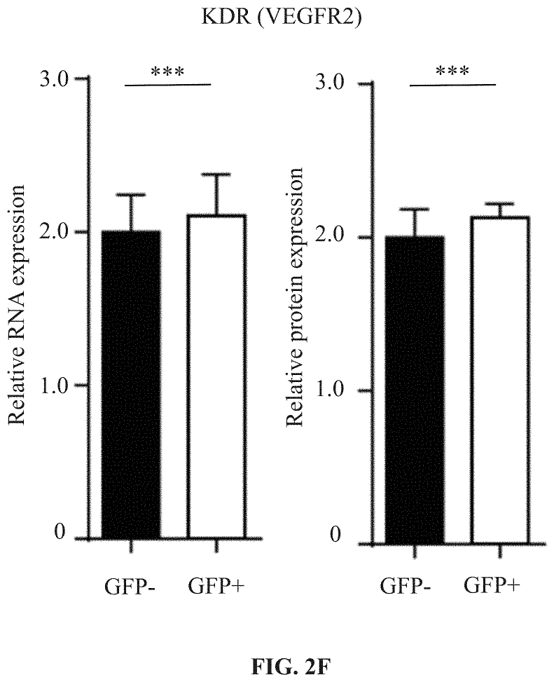

In an illustrative embodiment of the present invention, as a proof of concept, provided is a CLDN5 transcriptional reporter in wherein the reporter gene GFP is inserted at the 3′ end of the CLDN5 gene The reporter gene serves as a surrogate marker of endothelial cells of high barrier function, i.e. TBI (see FIG. 1A). The reporter hPSC line can be differentiated to ECs wherein, e.g., a 20% GFP+ population of ECs is generated. The cells can be further FACS sorted as described herein into the GFP+ and GFP− population wherein a significant increase in barrier resistance of GFP+ ECs compared to GFP− ECs is observed (see FIG. 2A and FIG. 2B).

As described herein, the expression of the reporter gene is operationally coupled to the expression of the tight junction protein. In an illustrative embodiment of the present invention, as a proof of concept, provided is a CLDN5 transcriptional reporter wherein the CLDN5 gene reporter is expressed as a fusion protein and subsequently processed to individual tight junction protein and reporter protein. The processing to individual proteins has the advantage that the tight junction gene, e.g., CLDN5, exert its cellular function without potential disturbance or disruption of interactions due to the attached reporter polypeptide. Accordingly, in a preferred embodiment of the invention, the tight junction gene reporter gene fusion protein is expressed and subsequently processed to individual separate proteins. The subsequent processing can for example be effected by introducing a self-cleaving peptide between the tight junction gene and the reporter gene.

However, other systems as known in the art are also useful to express the reporter gene and tight junction gene, preferably from the same gene locus, for example internal ribosomal entry sites (IRES) are used in the art to express two proteins from one promoter.

Accordingly, the method as described herein combines the generation of a EC population with high expression of (a) tight junction gene(s) to establish a cell culture model with high TBI, with a reporter function to assess the level of expression of (a) tight junction gene(s). This is particularly useful to establish standardized cell cultures for high-throughput screening, e.g., drug testing, assessing tissue barrier function in response to a drug. The measurement of the reporter gene, e.g., GFP can be used to establish the cell culture system for screening, and subsequently as a readout (assessable signal) during the screening process itself. Without being bound to theory, the expression of the tight junction gene(s), for which the introduced reporter gene is a surrogate marker, is indicative for integrity or breakdown of the barrier function, e.g., TBI.

In another variation of the invention, the TBI is directly measured by methods known in the art. In such embodiments of the invention, the reporter gene is used mainly or primarily to enrich the EC population for cells with high expression of the tight junction gene(s). The resulting enriched cell population can thereafter be used to establish the cell culture model of TBI. The measurement before and/or after application of the drug candidate is accomplished by a method directly assessing barrier function, for example transendothelial electrical resistance or FITC dextran mobility, or other measurements of barrier integrity or breakdown as well known in the art. Accordingly, in one particular embodiment, provided is the method as described herein, wherein step c) comprises measuring the transendothelial electrical resistance (TEER) wherein the measured TEER is indicative for TBI. A system capable of measuring the TEER in a high-throughput mode is for example the ECIS Z-theta system from Applied Biophysics wherein 96 well array plates can be used to establish the TEER in a drug-screening setup.

As described herein, the reporter gene is operationally coupled to a tight junction gene promoter, preferentially by integrating the reporter gene into the gene locus of the tight junction gene. In particular, the reporter gene can be integrated into the genome of the ECs by gene editing, for example using the CRISPR/CAS9 gene editing system. Tight junction genes are known in the art and can be further selected according to their expression pattern in EC populations establishing high resistance barrier function or failing to establish high resistance barrier function. Barrier function can be measured as described herein. In particular embodiments of the present invention the tight junction gene is selected from the group consisting of CLDN5, ocludin (OCLN) and MARVELD3, in particular wherein the tight junction gene is CLDN5.

The ECs provided in step a) of the methods of the present invention can be produced in vitro according to protocols known in the art. Particularly useful for the purpose of the present inventions are ECs deriving from pluripotent stem cell. Pluripotent stem cells have self-renewal character and can be differentiated in all major cell types of the adult mammalian body. Pluripotent stem cells are particularly useful for the method of the present invention because they can be produced in large quantities under standardized cell culture conditions. Accordingly, preferably, the ECs are differentiated from pluripotent stem cells. In one embodiment, the ECs are differentiated from embryonic stem cells. In another embodiment, the ECs are differentiated from induced pluripotent stem cells (IPSCs). In one embodiment the IPSCs are generated from reprogrammed somatic cells. Reprogramming of somatic cells to IPSCs can be achieved by introducing specific genes involved in the maintenance of IPSC properties. Genes suitable for reprogramming of somatic cells to IPSCs include, but are not limited to Oct4, Sox2, Klf4 and C-Myc and combinations thereof. In one embodiment the genes for reprogramming are Oct4, Sox2, Klf4 and C-Myc. Combinations of genes for transdifferentiating somatic cells to NPCs are described in WO2012/022725 which is herein included by reference.

Internal organs, skin, bones, blood and connective tissue are all made up of somatic cells. Somatic cells used to generate IPSCs include but are not limited to fibroblast cells, adipocytes and keratinocytes and can be obtained from skin biopsy. Other suitable somatic cells are leucocytes, erythroblasts cells obtained from blood samples or epithelial cells or other cells obtained from blood or urine samples and reprogrammed to IPSCs by the methods known in the art and as described herein. The somatic cells can be obtained from a healthy individual or from a diseased individual. In one embodiment, the somatic cells are derived from a subject (e.g., a human subject) suffering from a disease. In one embodiment, the disease is associated with vascular complications (e.g., similar to or identical to vascular complications associated with diabetic retinopathy and/or Wet AMD). The genes for reprogramming as described herein are introduced into somatic cells by methods known in the art, either by delivery into the cell via reprogramming vectors or by activation of said genes via small molecules. Methods for reprogramming comprise, inter alia, retroviruses, lentiviruses, adenoviruses, plasmids and transposons, microRNAs, small molecules, modified RNAs messenger RNAs and recombinant proteins. In one embodiment, a lentivirus is used for the delivery of genes as described herein. In another embodiment, Oct4, Sox2, Klf4 and C-Myc are delivered to the somatic cells using Sendai virus particles. In addition, the somatic cells can be cultured in the presence of at least one small molecule. In one embodiment, said small molecule comprises an inhibitor of the Rho-associated coiled-coil forming protein serine/threonine kinase (ROCK) family of protein kinases. Non-limiting examples of ROCK inhibitors comprise fasudil (1-(5-Isoquinolinesulfonyl) homopiperazine), Thiazovivin (N-Benzyl-2-(pyrimidin-4-ylamino) thiazole-4-carboxamide) and Y-27632 ((+)-(R)-trans-4-(1-aminoethyl)-N-(4-pyridyl) cyclo-hexanecarboxamide dihydrochloride).

Providing a defined monolayer of pluripotent stem cells is preferred for reproducibility and efficiency of the resulting cultures. In one embodiment, monolayers of pluripotent stem cells can be produced by enzymatically dissociating the cells into single cells and bringing them onto an adhesive substrate, such as pre-coated matrigel plates (e.g. BD Matrigel hESC-qualified from BD Bioscience, Geltrex hESC-qualified from Invitrogen, Synthemax from Corning). Examples of enzymes suitable for the dissociation into single cells include Accutase (Invitrogen), Trypsin (Invitrogen), TrypLe Express (Invitrogen). In one embodiment, 20000 to 60000 cells per cm2 are plated on the adhesive substrate. The medium used herein is a pluripotency medium which facilitates the attachment and growth of the pluripotent stem cells as single cells in a monolayer. In one embodiment, the pluripotency medium is a serum free medium supplemented with a small molecule inhibitor of the Rho-associated coiled-coil forming protein serine/threonine kinase (ROCK) family of protein kinases (herein referred to as ROCK kinase inhibitor).

Thus, in one embodiment, step a) of the method described above comprises providing a monolayer of pluripotent stem cells in a pluripotency medium, wherein said pluripotency medium is a serum free medium supplemented with a ROCK kinase inhibitor.

Examples of serum-free media suitable for the attachment of the pluripotent stem cells to the substrate are mTeSR1 or TeSR2 from Stem Cell Technologies, Primate ES/iPS cell medium from ReproCELL and StemPro hESC SFM from Invitrogen, X-VIVO from Lonza. Examples of ROCK kinase inhibitor useful herein are Fasudil (1-(5-Isoquinolinesulfonyl)homopiperazine), Thiazovivin (N-Benzyl-2-(pyrimidin-4-ylamino)thiazole-4-carboxamide) and Y27632 ((+)-(R)-trans-4-(1-aminoethyl)-N-(4-pyridyl) cyclo-hexanecarboxamide dihydrochloride, e.g. Catalogue Number: 1254 from Tocris bioscience). In one embodiment, the pluripotency medium is a serum free medium supplemented with 2-20 μM Y27632, preferably 5-10 μM Y27632. In another embodiment the pluripotency medium is a serum free medium supplemented with 2-20 μM Fasudil. In another embodiment the pluripotency medium is a serum free medium supplemented with 0.2-10 μM Thiazovivin.

In one embodiment step a) of the method described above comprises providing a monolayer of pluripotent stem cells in a pluripotency medium and growing said monolayer in the pluripotency medium for one day (24 hours). In another embodiment step a) of the method described above comprises providing a monolayer of pluripotent stem cells in a pluripotency medium and growing said monolayer in the pluripotency medium for 18 hours to 30 hours, preferably for 23 to 25 hours.

In another embodiment step a) of the method described above comprises providing a monolayer of pluripotent stem cells in a pluripotency medium, wherein said pluripotency medium is a serum-free medium supplemented with a ROCK kinase inhibitor, and growing said monolayer in the pluripotency medium for one day (24 hours). In another embodiment step a) of the method described above comprises providing a monolayer of pluripotent stem cells in a pluripotency medium, wherein said pluripotency medium is a serum-free medium supplemented with a ROCK kinase inhibitor, and growing said monolayer in the pluripotency medium for 18 hours to 30 hours, preferably for 23 to 25 hours.

In one embodiment the cells are contacted with a priming medium to induce differentiation. In one embodiment, the cells are contacted with a priming medium supplemented with a small molecule that activates the Beta-catenin and/or Wnt signaling and/or Hedgehog (HH) signaling and inducing differentiation by incubating the primed cells in an induction medium. In one embodiment, the small molecule that activates the Beta-catenin and/or Wnt signaling and/or Hedgehog (HH) signaling is selected from the group of small molecule inhibitors of glycogen synthase kinase 3 (Gsk3a-b), small molecule inhibitors of CDC-like kinase 1 (Clk1-2-4, small molecule inhibitors of mitogen-activated protein kinase 15 (Mapk15), small molecule inhibitors of dual-specificity tyrosine-(Y)-phosphorylation regulated kinase (Dyrk1a-b 4), small molecule inhibitors of cyclin-dependent kinase 16 (Pctk1-3 4), Smoothened (SMO) activators and modulators of the interaction between β-catenin (or γ-catenin) and the coactivator proteins CBP (CREB binding protein) and p300 (E1A binding protein p300).

Preferably said glycogen synthase kinase 3 (Gsk3a-b) inhibitors are pyrrolidindione-based GSK3 inhibitors. “Pyrrolidindione-based GSK3 inhibitor” as used herein relates to selective cell permeable ATP-competitive inhibitors of GSK3a and GSK3β with low IC₅₀ values. In one embodiment the pyrrolidindione-based GSK3 inhibitor is selected from the group consisting of SB216763 (3-(2,4-Dichlorophenyl)-4-(1-methyl-1H-indol-3-yl)-1H-pyrrole-2,5-dione), SB415286 (3-[(3-Chloro-4-hydroxyphenyl)amino]-4-(2-nitrophenyl)-1H-pyrrol-2,5-dione), N⁶-{2-[4-(2,4-Dichloro-phenyl)-5-imidazol-1-yl-pyrimidin-2-ylamino]-ethyl}-3-nitro-pyridine-2,6-diamine 2HCl, 3-Imidazo[1,2-a]pyridin-3-yl-4-[2-(morpholine-4-carbonyl)-1,2,3,4-tetrahydro-[1,4]diazepino[6,7,1-hi]indol-7-yl]-pyrrole-2,5-dione, Kenpaullone (9-Bromo-7,12-dihydro-indolo[3,2-d][1]benzazepin-6(5H)-one), CHIR99021 (9-Bromo-7,12-dihydro-pyrido[3′,2′:2,3]azepino[4,5-b]indol-6(5H)-one) and (3-(3-Amino-phenyl)-4-(1-methyl-1H-indol-3-yl)-pyrrole-2,5-dione (CP21R7, also referred to as “compound 21” herein; see e.g. L. Gong et al; Bioorganic & Medicinal Chemistry Letters 20 (2010), 1693-1696). In a preferred embodiment said pyrrolidindione-based GSK3 inhibitor is CP21R7.

In one embodiment said CDC-like kinase 1 (Clk1-2-4) inhibitor is selected from the group comprising benzothiazole and 3-Fluoro-N-[1-isopropyl-6-(1-methyl-piperidin-4-yloxy)-1,3-dihydro-benzoimidazol-(2E)-ylidene]-5-(4-methyl-1H-pyrazole-3-sulfonyl)-benzamide.

In one embodiment said mitogen-activated protein kinase 15 (Mapk15) inhibitor is selected from the group comprising 4-(4-Fluorophenyl)-2-(4-methylsulfinylphenyl)-5-(4-pyridyl)-1H-imidazole (SB203580) and 5-Isoquinolinesulfonamide (H-89).

In one embodiment said dual-specificity tyrosine-(Y)-phosphorylation regulated kinase (Dyrk1a-b 4) inhibitor is selected from the group comprising 6-[2-Amino-4-oxo-4H-thiazol-(5Z)-ylidenemethyl]-4-(tetrahydro-pyran-4-yloxy)-quinoline-3-carbonitrile.

In one embodiment said smoothened activator is Purmorphamine (2-(1-Naphthoxy)-6-(4-morpholinoanilino)-9-cyclohexylpurine.

Examples of modulators of the interaction between β-catenin (or γ-catenin) and the coactivator proteins CBP (CREB binding protein) and p300 (E1A binding protein p300) are IQ-1 (2-(4-Acetyl-phenylazo)-2-[3,3-dimethyl-3,4-dihydro-2H-isoquinolin-(1E)-ylidene]-acetamide, and ICG-001((6S,9aS)-6-(4-Hydroxy-benzyl)-8-naphthalen-1-ylmethyl-4,7-dioxo-hexahydro-pyrazino[1,2-a]pyrimidine-1-carboxylic acid benzylamide (WO 2007056593).

In one embodiment, the priming medium is supplemented with a small molecule inhibitor of Transforming growth factor beta (TGF β). In one embodiment, the small molecule inhibitor of TGF β is SB431542.

In one embodiment step a) of the method described above comprises incubating said cells in a priming medium for about 2 to about 4 days (about 48 hours to about 96 hours). In one embodiment, step a) of the method described above comprises incubating said cells in a priming medium for about 3 days (about 72 hours).

In one embodiment said priming medium is a serum free medium supplemented with insulin, transferrin and progesterone. In one embodiment said serum free medium is supplemented with 10-50 μg/ml insulin, 10-100 μg/ml transferrin and 10-50 nM progesterone, preferably 30-50 μg/ml insulin, 20-50 μg/ml transferrin and 10-30 nM progesterone. Examples of serum-free media suitable for priming are N2B27 medium (N2B27 is a 1:1 mixture of DMEM/F12 (Gibco, Paisley, UK) supplemented with N2 and B27 (both from Gibco)), N3 medium (composed of DMEM/F12 (Gibco, Paisley, UK), 25 μg/ml insulin, 50 μg/ml transferrin, 30 nM sodium selenite, 20 nM progesterone, 100 nM putrescine (Sigma)), or NeuroCult® NS-A Proliferation medium (Stemcell Technologies). In one embodiment said priming medium is a serum free medium supplemented with insulin, transferrin, progesterone and a small molecule that activates the Beta-Catenin (cadherin-associated protein, beta 1; human gene name CTNNB1) pathway and/or the Wnt receptor signaling pathway and/or hedgehog (HH) signaling pathway. Preferably said small molecule is selected from the group comprising 3-(2,4-Dichlorophenyl)-4-(1-methyl-1H-indol-3-yl)-1H-pyrrole-2,5-dione (SB216763), 3-[(3-Chloro-4-hydroxyphenyl)amino]-4-(2-nitrophenyl)-1H-pyrrol-2,5-dione (SB415286), N⁶-{2-[4-(2,4-Dichloro-phenyl)-5-imidazol-1-yl-pyrimidin-2-ylamino]-ethyl}-3-nitro-pyridine-2,6-diamine 2HCl, 3-Imidazo[1,2-a]pyridin-3-yl-4-[2-(morpholine-4-carbonyl)-1,2,3,4-tetrahydro-[1,4]diazepino[6,7,1-hi]indol-7-yl]-pyrrole-2,5-dione, 9-Bromo-7,12-dihydro-indolo[3,2-d][1]benzazepin-6(5H)-one (Kenpaullone), 9-Bromo-7,12-dihydro-pyrido[3′2′:2,3]azepino[4,5-b]indol-6(5H)-one (CHIR99021), 3-(3-Amino-phenyl)-4-(1-methyl-1H-indol-3-yl)-pyrrole-2,5-dione (CP21R7, also referred to as “compound 21” herein), benzothiazole, 3-Fluoro-N-[1-isopropyl-6-(1-methyl-piperidin-4-yloxy)-1,3-dihydro-benzoimidazol-(2E)-ylidene]-5-(4-methyl-1H-pyrazole-3-sulfonyl)-benzamide, 4-(4-Fluorophenyl)-2-(4-methylsulfinylphenyl)-5-(4-pyridyl)-1H-imidazole (SB203580), 5-Isoquinolinesulfonamide (H-89), 6-[2-Amino-4-oxo-4H-thiazol-(5Z)-ylidenemethyl]-4-(tetrahydro-pyran-4-yloxy)-quinoline-3-carbonitrile, 2-(1-Naphthoxy)-6-(4-morpholinoanilino)-9-cyclohexylpurine (Purmorphamine), 2-(4-Acetyl-phenylazo)-2-[3,3-dimethyl-3,4-dihydro-2H-isoquinolin-(1E)-ylidene]-acetamide (IQ-1), and ICG-001 ((6S,9aS)-6-(4-Hydroxy-benzyl)-8-naphthalen-1-ylmethyl-4,7-dioxo-hexahydro-pyrazino[1,2-a]pyrimidine-1-carboxylic acid benzylamide.

In another embodiment step a) of the method described above comprises incubating said cells in a priming medium, wherein said priming medium is a serum-free medium supplemented with CP21R7 (3-(3-Amino-phenyl)-4-(1-methyl-1H-indol-3-yl)-pyrrole-2,5-dione). Preferably said priming medium is supplemented with 0.5-4 μM CP21R7 (3-(3-Amino-phenyl)-4-(1-methyl-1H-indol-3-yl)-pyrrole-2,5-dione), most preferably 1-2 μM CP21R7 (3-(3-Amino-phenyl)-4-(1-methyl-1H-indol-3-yl)-pyrrole-2,5-dione). In another embodiment step a) of the method described above comprises incubating said cells in a priming medium, wherein said priming medium is a serum-free medium supplemented with CP21R7 (3-(3-Amino-phenyl)-4-(1-methyl-1H-indol-3-yl)-pyrrole-2,5-dione), and growing said cells for 2 to 4 days (48 hours to 96 hours). In another embodiment step a) of the method described above comprises incubating said cells in a priming medium, wherein said priming medium is a serum-free medium supplemented with CP21R7 (3-(3-Amino-phenyl)-4-(1-methyl-1H-indol-3-yl)-pyrrole-2,5-dione), and incubating said cells for three days (72 hours).

In one embodiment the priming medium is a serum-free medium containing 10-50 μg/ml insulin, 10-100 μg/ml transferrin and 10-50 nM progesterone supplemented with 0.5-4 μM CP21R7 (3-(3-Amino-phenyl)-4-(1-methyl-1H-indol-3-yl)-pyrrole-2,5-dione).

In one embodiment the priming medium additionally comprises recombinant bone morphogenic protein-4 (BMP4). In one preferred embodiment the priming medium is a serum-free medium containing 10-50 μg/ml insulin, 10-100 μg/ml transferrin and 10-50 nM progesterone supplemented with 0.5-4 μM CP21R7 (3-(3-Amino-phenyl)-4-(1-methyl-1H-indol-3-yl)-pyrrole-2,5-dione) and 10-50 ng/ml recombinant bone morphogenic protein-4 (BMP4).

In one embodiment the cells are contacted with an induction medium to proceed differentiation. For predominant induction of endothelial cells, said induction medium is supplemented with VEGF (=Vascular endothelial growth factor) or placenta-like growth factor 1 (PLGF-1) and a small molecule adenylate cyclase activator. In one embodiment said small molecule adenylate cyclase activator leads to the activation of PKA/PKI signaling pathway. In one embodiment, said small molecule adenylate activators are chosen from the group comprising Forskolin ((3R)-(6aalphaH)Dodecahydro-6beta,10alpha 10balpha-trihydroxy-3beta,4abeta,7,7,10 abeta-pentamethyl-1-oxo-3-vinyl-1H-naphtho[2,1-b]pyran-5beta-yl acetate), 8-Bromo-cAMP (8-Bromoadenosine-3′,5′-cyclic monophosphate) and Adrenomedullin. In one embodiment said induction medium is a serum free medium supplemented with human serum albumin, ethanolamine, transferrin, insulin and hydrocortisone. Examples of serum-free media suitable for the induction are StemPro-34 (Invitrogen, principal components: human serum albumin, lipid agents such as Human Ex-Cyte® and ethanolamine or a mixture thereof, human zinc insulin, hydrocortisone, iron-saturated transferring 2-mercaptoethanol, and D,L-tocopherol acetate, or derivatives or mixtures thereof) and X-VIVO 10 and 15 (Lonza).

In one embodiment, said induction medium is a serum-free medium supplemented with human serum albumin, ethanolamine, transferrin, insulin and hydrocortisone, and 1-10 μM Forskolin and 5-100 ng/ml VEGF-A. In another embodiment, the induction medium comprises StemPro-34 (from Invitrogen) supplemented with VEGF-A 30-70 ng/ml or placenta-like growth factor 1 (PLGF-1) 30-70 ng/ml.

In one embodiment step a) of the method described above comprises inducing the differentiation into endothelial cells by incubating said primed cells in an induction medium supplemented with VEGF-A or placenta-like growth factor 1 (PLGF-1) and a small molecule adenylate cyclase activator, wherein said small molecule adenylate cyclase activator is selected from the group of Forskolin, 8-Bromo-cAMP and Adrenomedullin. In one embodiment, the induction medium is a serum-free medium supplemented with 1-10 μM Forskolin and 5-100 ng/ml VEGF-A, preferably 2 μM Forskolin and 50 ng/ml VEGF-A

In another embodiment step a) of the method described above comprises inducing the differentiation into endothelial cells by incubating said primed cells in an induction medium supplemented with VEGF-A or placenta-like growth factor 1 (PLGF-1) and a small molecule adenylate cyclase activator for one day.

In another embodiment step a) of the method described above comprises inducing the differentiation into endothelial cells by incubating said primed cells in an induction medium supplemented with VEGF-A or placenta-like growth factor 1 (PLGF-1) and a small molecule adenylate cyclase activator for 18 hours to 48 hours, preferably for 22 hours to 36 hours.

In one embodiment, step a) of the method described above comprises incubating said cells the induction medium for about 18 hours to about 48 hours. In one embodiment step a) of the method described above comprises incubating said cells in an induction medium for about 24 hours.

After priming and induction, the ECs can be further expanded to produce large quantities of cells. Accordingly, in a further embodiment, said the method of the invention additionally comprises incubating the product of step a) under conditions suitable for proliferation of the endothelial cells. Preferably said conditions suitable for proliferation of the endothelial cells comprise harvesting of the cells positive for the reporter gene (e.g., GFP) and expanding them in a chemically defined expansion medium. “Harvesting” as used herein relates to the enzymatical dissociation of the cells from the adhesive substrate and subsequent resuspension in new medium. In one preferred embodiment, cells are sorted after harvesting as herein described. In one embodiment said expansion medium is a serum free medium supplemented with VEGF-A. Examples of serum-free media suitable for the expansion of endothelial cells are StemPro-34 (Invitrogen), EGM2 (Lonza) and DMEM/F12 (Invitrogen) supplemented with 8 ng/ml FGF-2, 50 ng/ml VEGF and 10 μM SB431542 (4-(4-Benzo[1,3]dioxol-5-yl-5-pyridin-2-yl-1H-imidazol-2-yl)-benzamide). Preferably, the endothelial cells are cultured in adherent culturing conditions. In one embodiment, the expansion medium is supplemented with 5-100 ng/ml VEGF-A. In another embodiment, the expansion medium is StemPro-34 supplemented with 5-100 ng/ml VEGF-A, preferably 50 ng/ml.

The ECs according to the present invention comprising a reporter gene under the control of a tight junction gene promoter can be enriched for cells expressing the reporter gene, which will be indicative for expression of the tight junction gene. Different cell sorting and enrichment protocols are known in the art. Examples of cell sorting methods include flow cytometry including fluorescence activated cell sorting (FACS) and magnetic activated cell sorting (MACS). In a preferred embodiment, the ECs express the reporter gene intracellularly, e.g. GFP. However, a reporter protein located partially or completely on the cell surface of the ECs is also envisaged, e.g., the reporter gene encodes for a transmembrane protein comprising an extracellular portion accessible for cell surface labelling and the respective sorting and enrichment technique (e.g., MACS). Flow cytometry analysis presented herein demonstrated that GFP positive cells in a culture can be enriched from less than 40% to up to 60% or more of the total cells, preferably, from less than 30% to up to 80% or more of the total cells, most preferably to up to more than 90% of the total cells. As herein described, the majority of cells in the GFP positive fraction showed typical EC morphology. In particular, the enriched fraction showed increased transendothelial electrical resistance (TEER).

The endothelial cells obtained by the method described herein can be expanded for several passages and culturing is well characterized. It is possible to freeze and thaw aliquots of the endothelial cells obtained by the method described herein reproducibly. Thawed cells can be further expanded as described herein to reach a desired number of cells which is particularly suitable to establish the throughput needed for compound screening.

The cells produced according to the methods of the present invention are useful to establish in vitro models of pathological or non-pathological conditions wherein the establishment or loss of transendothelial barrier function is of relevance. In a particular embodiment, provided is an in vitro method for identifying a drug candidate capable of i) increasing in vivo transendothelial barrier integrity (TBI) or ii) decreasing in vivo TBI of endothelial cells (ECs), the method consisting of the sequential the steps of:
a) providing ECs comprising a reporter gene under the control of a tight junction gene promoter, wherein the ECs are enriched for cells expressing the reporter gene;
b) contacting the ECs with the drug candidate and measuring in vitro TBI before and after contacting the ECs with the drug candidate, or contacting the ECs with the drug candidate and measuring in vitro TBI of the ECs contacted with the drug candidate and in parallel measuring in vitro TBI of ECs not contacted with the drug candidate;

wherein (i) a higher in vitro TBI of the ECs contacted with the drug candidate compared with the in vitro TBI of the ECs not contacted with the drug candidate is indicative of a drug capable of increasing in vivo TBI of ECs, and (ii) a lower in vitro TBI of the ECs contacted with the drug candidate compared with the in vitro TBI of the ECs not contacted with the drug candidate is indicative of a drug capable of decreasing in vivo TBI of ECs.

In a further embodiment, provided is an in vitro method for selecting a drug candidate for in vivo application to an individual suffering from a disease associated with disruption or loss of transendothelial barrier integrity (TBI), the method consisting of the sequential the steps of:
a) providing ECs comprising a reporter gene under the control of a tight junction gene promoter, wherein the ECs are enriched for cells expressing the reporter gene;
b) contacting the ECs with the drug candidate and measuring in vitro TBI before and after contacting the ECs with the drug candidate, or contacting the ECs with the drug candidate and measuring in vitro TBI of the ECs contacted with the drug candidate and in parallel measuring in vitro TBI of ECs not contacted with the drug candidate;

wherein a drug candidate with a higher in vitro TBI of the ECs contacted with the drug candidate compared with the in vitro TBI of the ECs not contacted with the drug candidate is selected for in vivo application of the drug candidate.

As herein described, the method of the present invention provides EC cultures with an increased yield of cells with increased tight junction formation and, accordingly, increased barrier integrity. In one embodiment, provided is a cell culture produced according to step a) of the in vitro method as described herein. The cell cultures as used and described herein are preferably enriched for ECs expressing the reporter gene as described herein. Accordingly, the cell cultures as used and described herein comprise more than 30%, 40%, 50%, 60%, 70%, 80%, 90%, 95% or more than 99% ECs expressing the reporter gene. In a preferred embodiment, the cell cultures as provided herein comprise more than 90% of ECs expressing the reporter gene, most preferably more than 95% of ECs expressing the reporter gene. Without being bound to theory, expression of the reporter gene will correlate with expression of the tight junction gene which controls the expression of the reporter gene. Accordingly, the present invention provides EC cell culture, wherein more than 30%, 40%, 50%, 60%, 70%, 80%, 90%, 95% or more than 99% ECs express the tight junction gene, e.g., CLDN5, OCLN and MARVELD3. In a preferred embodiment, the cell cultures as provided herein comprise more than 90% of ECs expressing CNDN5, most preferably more than 95% of ECs expressing CNDN5.

In the context of the present invention, higher in vitro TBI means that a higher value of a parameter correlating with TBI, e.g., TEER or expression of the reporter gene as herein described, is measured for a cell culture of interest (e.g., the EC culture contacted with a drug candidate) in comparison to a cell culture at reference conditions (e.g., the EC culture not contacted with a drug candidate). In one embodiment, the measured in vitro TBI of the EC culture contacted with the drug candidate is higher compared to the measured in vitro TBI of the EC culture not contacted with the drug candidate, in particular at least about 1.5-fold, 2-fold, 3-fold, 4-fold, 5-fold, or 10-fold higher compared to the measured in vitro TBI of the EC culture not contacted with the drug candidate. In one embodiment, the measured in vitro TBI of the EC culture contacted with the drug candidate is lower compared to the measured in vitro TBI of the EC culture not contacted with the drug candidate, in particular at least about 1.5-fold, 2-fold, 3-fold, 4-fold, 5-fold, or 10-fold lower compared to the measured in vitro TBI of the EC culture not contacted with the drug candidate. In one embodiment, step c) of the method as described herein comprises measuring the transendothelial electrical resistance (TEER) wherein the measured TEER is indicative for in vitro TBI. In one embodiment, the measured TEER of the EC culture contacted with the drug candidate is higher compared to the measured TEER of the EC culture not contacted with the drug candidate, in particular at least about 1.5-fold, 2-fold, 3-fold, 4-fold, 5-fold, or 10-fold higher compared to the TEER of the EC culture not contacted with the drug candidate. In one embodiment, the measured TEER of the EC culture contacted with the drug candidate is lower compared to the measured TEER of the EC culture not contacted with the drug candidate, in particular at least about 1.5-fold, 2-fold, 3-fold, 4-fold, 5-fold, or 10-fold lower compared to the TEER of the EC culture not contacted with the drug candidate. In one embodiment, the reporter gene is a fluorescent protein (e.g., GFP) and the measured fluorescence of ECs (e.g., the EC culture) contacted with the drug candidate is higher compared to the measured fluorescence of ECs (e.g., the EC culture) not contacted with the drug candidate, in particular at least about 1.5-fold, 2-fold, 3-fold, 4-fold, 5-fold, or 10-fold higher compared to the fluorescence of ECs (e.g., the EC culture) not contacted with the drug candidate. In one embodiment, the reporter gene is a fluorescent protein (e.g., GFP) and the measured fluorescence of ECs (e.g., the EC culture) contacted with the drug candidate is lower compared to the measured fluorescence of ECs (e.g., the EC culture) not contacted with the drug candidate, in particular at least about 1.5-fold, 2-fold, 3-fold, 4-fold, 5-fold, or 10-fold lower compared to the fluorescence of the ECs (e.g., the EC culture) not contacted with the drug candidate. Means for measuring TEER and fluorescence are well known in the art and also described herein.

In one embodiment of the present invention a method for generating patient specific or healthy individual specific ECs with high TBI is provided. This is particularly desirable for disease condition associated with a genetic mutation, however, a patient specific disease model can also be relevant where no genetic mutation is associated with the disease condition or in situations where a link to a genetic mutation is not known or should be established. Towards this end, human induced pluripotent stem cells (iPSCs) obtained from a patient or healthy individual are used in the method described herein. Said patient-specific human iPSCs can be obtained by methods known in the art and as further described herein by reprogramming somatic cells obtained from the patients or healthy individuals to pluripotent stem cells. For example, fibroblast cells, keratinocytes or adipocytes may be obtained by skin biopsy from the individual in need of treatment or from a healthy individual and reprogrammed to induced pluripotent stem cells by the methods known in the art and as further described herein. Other somatic cells suitable as a source for induced pluripotent stem cells are leucocytes cells obtained from blood samples or epithelial cells or other cells obtained from urine samples. The patient specific induced pluripotent stem cells are then differentiated to patient specific diseased or healthy ECs by the method described herein. In another aspect of the invention, a population of ECs produced by any of the foregoing methods is provided. Preferably, the population of ECs is patient specific, i.e. derived from iPSCs obtained from diseased individuals. In another embodiment said population of ECs is obtained from a healthy individual. Patient derived ECs represent a disease relevant in vitro model to study the pathophysiology of vascular complications for diseases like Diabetes Type-2 and Type-1, Wet AMD, Metabolic Syndrome and Severe Obesity. In one embodiment the ECs obtained by this method are used for screening for compounds that reverse, inhibit or prevent vascular complications caused by dysfunction of endothelial cells, e.g. of vascular complications caused by diabetes Type-2 and Type-1, Wet AMD, Metabolic Syndrome, Severe Obesity, Hypercholesterolemia, Hypertension, coronary artery disease, nephropathy, retinopathy, kidney failure, tissue ischemia, chronic hypoxia, artherosclerosis and tissue edema caused by drug-induced toxicity. Preferably, said ECs obtained by the method of the invention described herein are derived from diseased subjects. Differentiating ECs from diseased subjects represents a unique opportunity to early evaluate drug safety in a human background paradigm.

In another embodiment the ECs obtained by this method are used as an in vitro model of the blood-retinal barrier (BRB) and/or the blood brain barrier (BBB).

One embodiment is the use of the EC cultures obtained by the methods according to the invention to determine the efficacy of a drug candidate. The cultures can be derived from healthy individuals and/or from diseased individuals and results from efficacy and/or toxicity studies performed using the EC cultures as described herein can be integrated to predict disease and/or therapy relevant physiological effects of a drug candidate. In one embodiment, the in vitro efficacy profile of a drug candidate is assessed and drug candidates with favorable efficacy profile are selected for further development. Further development may comprise in vivo testing of the drug candidate in non-human primate species and/or in vivo testing in humans.

### EXEMPLARY EMBODIMENT

1. An in vitro method for identifying a drug candidate capable of i) increasing in vivo transendothelial barrier integrity (TBI) or ii) decreasing in vivo TBI of endothelial cells (ECs) comprising the steps of:
   a) providing ECs comprising a reporter gene under the control of a tight junction gene promoter, in particular wherein the ECs are enriched for cells expressing the reporter gene;
   b) contacting the ECs with the drug candidate;
   c) measuring in vitro TBI before and after contacting the ECs with the drug candidate, or measuring in vitro TBI of the ECs contacted with the drug candidate and in parallel measuring in vitro TBI of ECs not contacted with the drug candidate;
   wherein (i) a higher in vitro TBI of the ECs contacted with the drug candidate compared with the in vitro TBI of the ECs not contacted with the drug candidate is indicative of a drug capable of increasing in vivo TBI of ECs, and (ii) a lower in vitro TBI of the ECs contacted with the drug candidate compared with the in vitro TBI of the ECs not contacted with the drug candidate is indicative of a drug capable of decreasing in vivo TBI of ECs.
2. The method of embodiment 1, wherein the ECs in step a) are provided as a monolayer of cells, in particular as a confluent monolayer of cells.
3. The method of any one of embodiments 1 or 2, wherein the ECs in step a) are provided on a cell culture support, in particular on a multi-well plate, more particular on a multi-well plate selected from the group consisting of a 24-well plate, a 96-well plate, a 384-well plate, or a 1536-well plate.
4. The method of any one of embodiments 1 to 3, wherein step c) comprises measuring the transendothelial electrical resistance (TEER) wherein the measured TEER is indicative for in vitro TBI.
5. The method of any one of embodiments 1 to 3, wherein step c) comprises measuring the expression of the reporter gene wherein the expression of the reporter gene is indicative for in vitro TBI.
6. The method of any one of embodiments 1 to 5, wherein the tight junction gene is selected from the group consisting of CLDN5, ocludin (OCLN) and MARVELD3, in particular wherein the tight junction gene is CLDN5.
7. The method any one of embodiments 1 to 6, wherein the ECs are differentiated from pluripotent stem cells.
8. The method of any one of embodiments 1 to 7, wherein the pluripotent stem cells are embryonic stem cells or induced pluripotent stem cells.
9. The method of any one of embodiments 1 to 8, wherein the pluripotent stem cell are human cells.
10. The method of any one of embodiments 1 to 9, wherein the pluripotent stem cells are derived from a subject suffering from a disease associated with vascular complications.
11. The method of any one of embodiments 7 to 10, wherein step a) comprises incubating the pluripotent stem cells in a priming medium supplemented with a small molecule that activates the Beta-catenin and/or Wnt signaling and/or Hedgehog (HH) signaling and inducing differentiation by incubating the primed cells in an induction medium.
12. The method of embodiment 11, wherein the small molecule that activates the Beta-catenin and/or Wnt signaling and/or Hedgehog (HH) signaling is selected from the group consisting of small molecule inhibitors of glycogen synthase kinase 3 (Gsk3a-b), small molecule inhibitors of CDC-like kinase 1 (Clk1-2-4, small molecule inhibitors of mitogen-activated protein kinase 15 (Mapk15), small molecule inhibitors of dual-specificity tyrosine-(Y)-phosphorylation regulated kinase (Dyrk1a-b 4), small molecule inhibitors of cyclin-dependent kinase 16 (Pctk1-3 4), Smoothened (SMO) activators and modulators of the interaction between β-catenin (or γ-catenin) and the coactivator proteins CBP (CREB binding protein) and p300 (E1A binding protein p300).
13. The method of any one of embodiments 11 or 12, wherein the priming medium is supplemented with a small molecule inhibitor of Transforming growth factor beta (TGF β).
14. The method of embodiment 13, wherein the small molecule inhibitor of TGF β is SB431542.
15. The method of any one of embodiments 11 to 14, wherein step a) comprises incubating the cells in the priming medium for 2 to 4 days, in particular for 3 days.
16. The method of any one of embodiments 11 to 15, wherein the priming medium of step a) is a serum free medium supplemented with insulin, transferrin and progesterone.
17. The method of any one of embodiments 11 to 16, wherein the small molecule that activates the Beta-catenin and/or Wnt signaling and/or Hedgehog (HH) signaling of step a) is 3-(3-Amino-phenyl)-4-(1-methyl-1H-indol-3-yl)-pyrrole-2,5-dione (CP21R7).
18. The method of any one of embodiments 11 to 17, wherein the priming medium of step a) additionally comprises recombinant bone morphogenic protein-4 (BMP4).
19. The method of any one of embodiments 11 to 18, wherein the priming medium is a serum-free medium containing 10-50 μg/ml insulin, 10-100 μg/ml transferrin and 10-50 nM progesterone supplemented with 0.5-4 μM CP21R7 (3-(3-Amino-phenyl)-4-(1-methyl-1H-indol-3-yl)-pyrrole-2,5-dione) and 10-50 ng/ml recombinant bone morphogenic protein-4 (BMP4), in particular wherein the priming medium comprises 1 μM CP21R7 and 25 ng/ml BMP4.
20. The method of any of embodiments 11 to 19, wherein the induction medium is a serum-free medium supplemented with VEGF-A (Vascular endothelial growth factor) or placenta-like growth factor 1 (PLGF-1) and a small molecule adenylate cyclase activator.
21. The method of embodiment 20, wherein the small molecule adenylate activators is selected from the group comprising Forskolin 43R)-(6aalphaH)Dodecahydro-6beta,10alpha,10balpha-trihydroxy-3beta,4abeta,7,7,10abeta-pentamethyl-1-oxo-3-vinyl-1H-naphtho[2,1-b]pyran-5beta-yl acetate), 8-Bromo-cAMP (8-Bromoadenosine-3′,5′-cyclic monophosphate) and Adrenomedullin.
22. The method of any one of embodiments 11 to 21, wherein the induction medium is a serum-free medium supplemented 1-10 μM Forskolin and 5-100 ng/ml VEGF-A, in particular 200 ng/ml VEGF and 2 μM Forskolin.
23. The method of any one of embodiments 11 to 22, wherein step a) comprises incubating the cells in the induction medium for 18 hours to 48 hours.
24. The method of any one of embodiments 1 to 23, additionally comprising incubating the product of step a) in an expansion medium suitable for proliferation of the ECs.
25. The method of embodiment 24, wherein the expansion medium is supplemented with VEGF-A, in particular with 50 ng/ml VEGF-A.
26. The method of any one of embodiments 1 to 25 wherein a polynucleotide encoding the reporter gene is inserted at the 3′ end of the tight junction gene, in particular wherein (i) a tight junction gene reporter gene fusion protein is expressed or (ii) the reporter gene is expressed from an internal ribosomal entry site (IRES), or (iii) a tight junction gene reporter gene fusion protein is expressed and subsequently processed to individual tight junction protein and reporter protein.
27. The method of embodiment 26(iii), wherein a polynucleotide encoding a self-cleaving peptide is introduced between the tight junction gene and the reporter gene, in particular wherein the self-cleaving peptide is the P2A self-cleaving peptide.
28. The method of any one of embodiments 1 to 27, wherein activation of the promoter of the tight junction gene leads to expression of the reporter gene.
29. The method of any one of embodiments 1 to 28, wherein the reporter gene encodes a luminescent protein, in particular a fluorescent protein.
30. The method of any one of embodiments 1 to 29, wherein the reporter gene encodes green fluorescent protein (GFP).
31. The method of any one of embodiment 1 to 30, wherein the cells are enriched for cells expressing the reporter gene in step a) by fluorescence activated cell sorting (FACS) or magnetic activated cell sorting (MACS).
32. The method of embodiment 24 to 31, wherein the cells are enriched for cells expressing the reporter gene before contacting the cells with the expansion medium.
33. The method of any one of embodiments 1 to 32, which is performed in a high-throughput format.
34. The method of any one of embodiments 1 to 33, which is used to screen molecules in a drug development setting, in particular for high-throughput screening a drug candidate compound library.
35. A cell culture produced according to step 1) a) of any one of embodiments 1 to 34, in particular wherein the percentage of cells expressing the tight junction gene is higher than 30%, 40%, 50%, 60%, 70%, 80%, 90%, or 95%.
36. A cell capable of expressing a reporter gene, wherein expression of the reporter gene is under the control of the promoter of a tight junction gene.
37. The cell of embodiment 36, wherein the cell comprises a polynucleotide encoding the reporter gene, wherein the polynucleotide encoding the reporter gene is inserted at the 3′ end of a tight junction gene.
38. The cell of any one of embodiment 36 or 37, wherein the cell comprises a polynucleotide encoding (i) a tight junction gene reporter gene fusion protein or (ii) a self-cleaving peptide between the tight junction gene and the reporter gene, in particular wherein the self-cleaving peptide is the P2A self-cleaving peptide.
39. The cell of any one of embodiments 36 to 38, wherein activation of the promoter of the tight junction gene leads to expression of the reporter gene.
40. The cell of any one of embodiments 36 to 39, wherein the tight junction gene is selected from the group consisting of CLDN5, ocludin (OCLN) and MARVELD3, in particular wherein the tight junction gene is CLDN5.
41. The cell of any one of embodiments 36 to 40, wherein the reporter gene is coding for a luminescent protein, in particular wherein the reporter gene is coding for a fluorescent protein, more particular wherein the reporter gene is coding for green fluorescent protein (GFP).
42. 2-[3-(6-Methyl-2-pyridinyl)-1H-pyrazol-4-yl]-1,5-naphthyridine for use in the treatment of a disease associated with vascular complications.
43. 4-[4-[3-(2-Pyridinyl)-1H-pyrazol-4-yl]-2-pyridinyl]-N-(tetrahydro-2H-pyran-4-yl)-benzamide for use in the treatment of a disease associated with vascular complications.
44. 2-[3-(6-Methyl-2-pyridinyl)-1H-pyrazol-4-yl]-1,5-naphthyridine for use according to embodiment 42, wherein the disease is selected from the group consisting of diabetes Type-2 and Type-1, diabetic retinopathy, Wet AMD, Metabolic Syndrome, Severe Obesity, Hypercholesterolemia, Hypertension, coronary artery disease, nephropathy, retinopathy, kidney failure, tissue ischemia, chronic hypoxia, artherosclerosis and tissue edema caused by drug-induced toxicity.
45. 4-[4-[3-(2-Pyridinyl)-1H-pyrazol-4-yl]-2-pyridinyl]-N-(tetrahydro-2H-pyran-4-yl)-benzamide for use according to embodiment 43, wherein the disease is selected from the group consisting of diabetes Type-2 and Type-1, diabetic retinopathy, Wet AMD, Metabolic Syndrome, Severe Obesity, Hypercholesterolemia, Hypertension, coronary artery disease, nephropathy, retinopathy, kidney failure, tissue ischemia, chronic hypoxia, artherosclerosis and tissue edema caused by drug-induced toxicity.
46. 2-[3-(6-Methyl-2-pyridinyl)-1H-pyrazol-4-yl]-1,5-naphthyridine for use according to embodiment 44, wherein the disease is diabetic retinopathy or Wet AMD.
47. 4-[4-[3-(2-Pyridinyl)-1H-pyrazol-4-yl]-2-pyridinyl]-N-(tetrahydro-2H-pyran-4-yl)-benzamide for use according to embodiment 45, wherein the disease is diabetic retinopathy or Wet AMD.
48. Use of 2-[3-(6-Methyl-2-pyridinyl)-1H-pyrazol-4-yl]-1,5-naphthyridine for the manufacture of a medicament for the treatment of a disease associated with vascular complications.
49. Use of 4-[4-[3-(2-Pyridinyl)-1H-pyrazol-4-yl]-2-pyridinyl]-N-(tetrahydro-2H-pyran-4-yl)-benzamide for the manufacture of a medicament for the treatment of a disease associated with vascular complications.
50. The use of any one of embodiments 48 or 49, wherein the disease is selected from the group consisting of diabetes Type-2 and Type-1, diabetic retinopathy, Wet AMD, Metabolic Syndrome, Severe Obesity, Hypercholesterolemia, Hypertension, coronary artery disease, nephropathy, retinopathy, kidney failure, tissue ischemia, chronic hypoxia, artherosclerosis and tissue edema caused by drug-induced toxicity.
51. The use of embodiment 50, wherein the disease is diabetic retinopathy or Wet AMD.
52. A method of treating a disease in an individual, comprising administering to said individual a therapeutically effective amount of a composition comprising 2-[3-(6-Methyl-2-pyridinyl)-1H-pyrazol-4-yl]-1,5-naphthyridine in a pharmaceutically acceptable form.
53. A method of treating a disease in an individual, comprising administering to said individual a therapeutically effective amount of a composition comprising 4-[4-[3-(2-Pyridinyl)-1H-pyrazol-4-yl]-2-pyridinyl]-N-(tetrahydro-2H-pyran-4-yl)-benzamide in a pharmaceutically acceptable form.
54. The method of any one of embodiments 52 or 53, wherein said disease is selected from the group consisting of diabetes Type-2 and Type-1, diabetic retinopathy, Wet AMD, Metabolic Syndrome, Severe Obesity, Hypercholesterolemia, Hypertension, coronary artery disease, nephropathy, retinopathy, kidney failure, tissue ischemia, chronic hypoxia, artherosclerosis and tissue edema caused by drug-induced toxicity.
55. The method of claim 54, wherein the disease is diabetic retinopathy or Wet AMD.
56. The invention as hereinbefore described.

### Materials and Methods

Human PSC culture and differentiation. The human ESC line SA001 (Zetterqvist A V, Blanco F, Ohman J, Kotova O, Berglund L M, de Frutos Garcia S, et al. Journal of diabetes research. 2015; 2015:428473.) was obtained from Cellartis AB (Englund M C, Caisander G, Noaksson K, Emanuelsson K, Lundin K, Bergh C, et al. In vitro cellular & developmental biology Animal. 2010; 46(3-4):217-30.). The cell line was routinely tested for mycoplasma contamination and was negative throughout this study. SA001 line has been tested with STR analysis, g-banding and Illumina SNP array (Omni Express). Upon CLDN5-P2A-GFP insertion, STR analysis, g-banding and Illumina SNP array (Omni Express) was repeated. Cells were routinely passaged using Accutase (StemCell Technologies) and replated as small clumps of cells at a dilution of 1:10 to 1:15. For differentiation, hPSCs were dissociated using Accutase. Differentiation protocol has been followed as described (Patsch C, Challet-Meylan L, Thoma E C, Urich E, Heckel T, O'Sullivan J F, et al. Nature cell biology. 2015; 17(8):994-1003.) with some modifications as follows: expansion medium consisting of StemPro with 50 ng/mL of VEGFA has been kept on cells only for the first division. From the second division cells were cultured using VascuLife VEGF Endothelial Medium Complete Kit (LifeLine Cell Technology). Final composition of the supplements added to the media was 10% FBS, 4 mM L-Glutamine, 0.75 U/mL Heparin sulfate, 5 ng/mL FGF-2, 5 ng/mL EGF, 5 ng/mL VEGFA, 15 ng/mL IGF1, 1 μg/mL Hydrocortizone Hemisuccinate, 50 μg/mL Ascorbic acid. SB431542 (10 μM) was supplemented to the media. The media was changed every other day. Experiments were performed with cells from passage 5 to passage 9.

Traceless integration of GFP reporter in CLDN5 locus using CRISPR/Cas9 genome editing and Piggybac excision. Cas9 targeting site was chosen close to the stop codon (GCGAGGCGTTGGATAAGCCT (SEQ ID NO: 1)), complementary sgRNA was produced by in vitro transcription (Thermo Fisher). The vector construct (FIG. 1B) for GFP integration by homologous recombination repair after CRISPR/Cas9 DNA double strand break was designed with homology arms flanking the left and right side of the stop codon of human CLDN5 by 694 and 518 bp lengths. The ATAA site, 61 nucleotides downstream of the stop codon of CLDN5 was changed into a TTAA in the right homologous recombination arm to allow further piggyBac excision of the resistance cassette. The vector carried resistances cassette for puromycin and truncated thymidine kinase under the EF1A promoter. Inverted terminal repeat (ITR) sequences allowing piggyBac excision and LoxP sites allowing Cre recombinase excision were present for the removal of the resistance cassette. The hPSCs were pretreated with 10 μM of Y-27632 (Calbiochem), 4 h before nucleofection. 200,000 cells were nucleofected using Amaxa 4d nucleofector (Lonza) with Primary cells P3 nucleofector solution (Lonza) using the CM130 program with 10.8 μg of specific sgRNA, 8 μg of Cas9 and 2.4 μg of plasmid vector donor. After nucleofection the cells were treated with 10 μM of Y-27632 for 24 h. Cells were left to recover from nucleofection for 5 days and then expanded under selection with puromycin (200 μg/mL). After selection, cells were nucleofected using Amaxa 4d nucleofactor (program: CM130) with excision-only piggyBac mRNA transposase (1.75 ug, Transposagen). Nucleofected cells were seeded, in serial dilution ranging from 1-300 cell/cm², on several culture plates. Single cell colonies that were well separated were picked after reaching 200 μm of diameter. Cells were washed with PBS and left in 0.1 mL/cm² PBS while picking the colonies. Colonies were detached by scratching off the colony with a sterile pipette tip and pipetting the colony and replating it on a matrigel coated 48-well plate with mTeSR1 medium. After 4 h medium was replaced by new mTeSR1 medium and further treated with 10 μM Y-27632 for 24 h.

Cells were expanded in mTeSR1 until confluency. DNA was isolated using BioSprint 96 DNA Blood Kit (Qiagen). Excision of resistance cassette was evaluated by qPCR using primers designed in the TK coding sequence (fwr-GTACCCGAGCCGATGACTTAC (SEQ ID NO: 2), rev-CCCGGCCGATATCTCA (SEQ ID NO: 3), probe-CTTCCGAGACAATCGCGAACATCTACACC (SEQ ID NO: 4)) and performed in a multiplex reaction with the reference gene RPP30 (fwr-GATTTGGACCTGCGA (SEQ ID NO: 5), rev-GCGGCTGTCTCCACA (SEQ ID NO: 6), probe-CTGACCTGAAGGCTCT (SEQ ID NO: 7)). QPCR was performed on the Light Cyler 480 (Roche) with the light cycler Kit (Roche) according to the manufacturer's instructions. Clones showing lowest expression of TK by qPCR were validated by PCR using FastStart kit (Roche) with primers that bind to GFP or TK or outside of the insert (R1-GGCTGGACAGAGAACAGGAC (SEQ ID NO: 8), F2-GCCCCCGAACCTTCAAAGA (SEQ ID NO: 9), R2-CTGCACGCCGTAGGTCAG (SEQ ID NO: 10), F3-GGAGATGGGGGAGGC TAACT (SEQ ID NO: 11)). Generated PCR products were run on a gel consisting of 1% agarose (Sigma) in TBE buffer (Life Technologies). PCR product was purified with the QIAquick PCR purification kit (Qiagen) according to manufacturer's instructions and sent for Sanger sequencing (Microsynth).

Fluorescence activated cell sorting and analysis. Human PSC-EC were dissociated from plates by Accutase (StemCell Technologies) and filtered before sorting with 30 μm filters (Miltenyi Biotec). Dissociated cells were kept in full media during sorting and sorted in cooled collection tubes with complete EC media supplemented with 20% FBS and 25 mM HEPES. Sorting was performed with BD FACS ARIA III (BD Biosciences) using 4-way purity precision mode. FACS plots were generated by Flowjo_V10 software. In case of RNA-seq minimum of 100,000 cells was sorted. After sorting, cells were spun down (610 g, 10 min) and lysed in 650 μL RLT lysis buffer (Qiagen)+β-mercaptoethanol (1%) and subsequently vortexed for 1 min at room temperature and snap frozen. In case of mass spectrometry analysis minimum of 10⁶ cells was sorted, cells were washed with PBS⁻(Life Technologies), spun down (110 g, 3 min), PBS was removed and cell pellets snap frozen.

RNA isolation. RNA isolation from FACS sorted or cultured cells was performed using RNeasy micro kit or RNeasy mini kit (both Qiagen) or automated Maxwell Total RNA purification kit (Promega), all procedures included DNAse I digestion. Procedures were followed as described in the kit protocols.

RNA-sequencing and analysis. Total RNA from the FACS sorted or cell cultured treated samples was subjected to oligo (dT) capture and enrichment, and the resulting mRNA fraction was used to construct complementary DNA libraries. Transcriptome sequencing (RNA-seq) was performed on the Illumina HiSeq platform using the standard protocol (TruSeq Stranded Total RNA Library, Illumina) that generated approximately 30 million reads of 50 base-pair per sample. FACS sorted experiments for GFP+ and GFP− cells were performed using 6 replicates each from 2 different clones. The RNA-seq reads were then mapped to the human genome (NCBI build 37) by using GSNAP (Wu T D, Nacu S. Bioinformatics (Oxford, England). 2010; 26(7):873-81.). Comparison was done between 12 samples of GFP+ and GFP− samples from two clones. TGFBR2 inhibitor treated hPSC-EC samples were mapped to the human genome (hg19/Refseq) using STAR (Dobin A, Davis Calif., Schlesinger F, Drenkow J, Zaleski C, Jha S, et al. Bioinformatics (Oxford, England). 2013; 29(1):15-21.) and counting was performed using union mode of HtSeq (Anders S, Pyl P T, Huber W. Bioinformatics (Oxford, England). 2015; 31(2):166-9.). Differential expression was performed using Deseq2 (Dobin A, Davis C A, Schlesinger F, Drenkow J, Zaleski C, Jha S, et al. Bioinformatics (Oxford, England). 2013; 29(1):15-21.). Gene set enrichment analysis was performed using GSEA (Subramanian A, Tamayo P, Mootha V K, Mukherjee S, Ebert B L, Gillette M A, et al. Proceedings of the National Academy of Sciences of the United States of America. 2005; 102(43):15545-50.) using Hallmarks MsigDb database (Liberzon A, Birger C, Thorvaldsdottir H, Ghandi M, Mesirov J P, Tamayo P. Cell systems. 2015; 1(6):417-25.) using weighted p2 analysis, following default conditions with ignoring gene sets smaller than 15 and larger than 5000 genes.

Compound library screening with fluorescence activated cell readout. Compounds were plated in replicate plates, and each plate had DMSO treated controls. ECs (10,000 cells per well) were seeded on fibronectin coated plates in full media. Cells were treated 2 days after seeding and the FACS measurement using MACS quant analyzer 10 (Miltenyi biotec) was performed. Screen data was analyzed in Flowjo_V10 software.

Electric cell-substrate impedance sensing. TBI was detected in real time using the ECIS® Z-theta system (Applied Biophysics, McAlister G C, Nusinow D P, Jedrychowski M P, Wuhr M, Huttlin E L, Erickson B K, et al. Analytical chemistry. 2014; 86(14):7150-8.) using 96 well array plates (96widf, Applied Biophysics) at 250 Hz frequency. Plates were coated with 100 μL of fibronectin (25 μg/mL; for 30 min at RT), fibronectin was replaced by complete media and electrodes were stabilized for 1 h on the system. Afterwards, media was removed and hPSC-ECs were seeded (10,000 cells per well). Cells were left for 2 days to reach full confluency and then treated with compounds with or without VEGFA (50 ng/mL). All the treatments were performed in triplicates.

FITC-dextran permeability assay. ECs were seeded on fibronectin coated transwell 96 well plates (Corning) in complete media. In bottom chamber 325 μL, and top 75 μl of EC media was added. Cells were left 2 days to attach and generate confluent monolayer. Cells were treated in the upper chamber with compounds and with or without VEGFA (50 ng/mL).

Competitive protein kinase binding assays. Kinome scan (468 kinases) was performed by scanMAX (DiscoveRx) at 1 μM. Kinome scan procedure has been followed as described in (Bemas M J, Cardoso F L, Daley S K, Weinand M E, Campos A R, Ferreira A J, et al. Nature protocols. 2010; 5(7):1265-72.). Kd determination for ACVR1B, TGFRB1, TGFBR2 and KDR has been done in duplicates with 11-point 3-fold serial dilution starting from 30 μM (DiscoveRx). All the compounds were dissolved in DMSO. Binding constants (Kds) were calculated with a standard dose-response curve using the Hill equation and the The Hill Slope was set to −1. Curves were fitted using a non-linear least square fit with the Levenberg-Marquardt algorithm.

Statistical analysis. Prism 7 (Graphpad) was used to create charts and perform statistical analyses. Statistical analysis was performed by unpaired, two-tailed Student's t-test, if not mentioned otherwise. For all bar graphs, data are represented as mean±SD. P values <0.05 were considered significant.

### Example 1

Genome Editing of the CLDN5 Transcriptional Reporter in hPSCs.

To evaluate the barrier properties of endothelial cells with a surrogate marker CLDN5 was tagged at the 3′ end with P2A self-cleaving peptide and GFP (FIG. 1A). We have designed the sgRNA in the vicinity of stop codon of CLDN5 while a donor plasmid (FIG. 1B) was generated to carry a promoterless P2A-GFP sequence flanked by two homology arms (HAs) at each end with piggyBac inverted terminal repeats (ITR) that allow traceless excision of the resistance cassettes. The double stranded break made by Cas9 and sgRNA was repaired by homologous recombination between CLDN5 and donor template (FIG. 1C) and subsequently resistance cassette was removed by excision only piggybac transposase (FIG. 1D). Single cell clones were picked and expanded.

We have evaluated lack of tTK by qPCR (not shown) and identified several clones lacking tTK. These clones were evaluated in gel PCR for correct insertion and orientation of GFP (FIG. 1E, F3/R1) and lack of tTK (FIG. 1E, F1/R1). The correct in-frame integration was confirmed by Sanger sequencing (FIG. 1F).

### Example 2

Generation and characterization of Stem-cell derived endothelial cells CLDN5 reporter. Using a previously published protocol (Patsch C, Challet-Meylan L, Thoma E C, Urich E, Heckel T, O'Sullivan J F, et al. Nature cell biology. 2015; 17(8):994-1003.) human pluripotent stem-cell line reporter line and WT line were differentiated to endothelial cells and 15-25% (FIG. 2A, depending on clone, data shown for one clone) of GFP+ cells and no GFP+ cells in WT line were observed. The GFP+ and GFP− cells were FACS-sorted and Electric Cell-substrate Impedance Sensing measurement was performed. An 1.75 fold increase of barrier resistance was observed in GFP+ cells (Resistance of 3200Ω, FIG. 2B). Next, RNA-sequencing and TMT mass proteomics was performed (not shown) on both GFP+ and GFP− FACS sorted cells and a very good correlation between significantly changed proteins and corresponding mRNAs was observed (r=0.79, p<0.0001, FIG. 2C). Significant upregulation of CLDN5 on mRNA and protein level (FIG. 2D) was confirmed, but also of other tight junction ocludin (OCLN) and MARVELD3 (FIG. 2E). Moreover, significant upregulation of adherans junctions CD31 was found. No difference in expression in VEGFR2 (KDR) or CD34 expression was observed suggesting that the difference in GFP+ and GFP− cells is in the barrier properties (FIG. 2F).

### Example 3

CLDN5-GFP+ ECs show functional response of high transendothelial barrier integrity. Next, gene-set enrichment analysis was performed (GSEA, Subramanian A, Tamayo P, Mootha V K, Mukherjee S, Ebert B L, Gillette M A, et al. Proceedings of the National Academy of Sciences of the United States of America. 2005; 102(43):15545-50.) with the Hallmarks MsigDB (Liberzon A, Birger C, Thorvaldsdottir H, Ghandi M, Mesirov J P, Tamayo P. Cell systems. 2015; 1(6):417-25.) database using the ranked list of a product of the log 2FC and the −log 10FDR of the comparison of GFP+ and GFP− sorted cells (data not shown). Interestingly, enrichment of angiogenesis, TGFß and E2F proliferation pathway was found among downregulated genes and enrichment in WNT signaling in upregulated gens (data not shown). The pathway enrichment analysis (Zhou Y, Wang Y, Tischfield M, Williams J, Smallwood P M, Rattner A, et al. The Journal of clinical investigation. 2014; 124(9):3825-46., Suzuki E, Nagata D, Yoshizumi M, Kakoki M, Goto A, Omata M, et al. The Journal of biological chemistry. 2000; 275(5):3637-44) confirmed that the GFP+ show a higher endothelial cell barrier properties. Next, GFP+ cell populations were treated with vascular endothelial growth factor (VEGFA), the most potent vascular permeability factor in vivo and striking loss of barrier properties was observed (FIG. 3A) and interestingly moreover the reduction in GFP+ cells in VEGFA treated conditions was observed. In the following experiment a broad tyrosine kinase receptor inhibitor SU11248 was used (Mendel D B, Laird A D, Xin X, Louie S G, Christensen J G, Li G, et al. Clinical cancer research. 2003; 9(1):327-37., PDGFR, VEGFR, c-Kit) and a striking increase in % of GFP+ cells was observed (99%, FIG. 3B). Intriguingly, treated cells were resistant to barrier breakdown by VEGFA as shown by ECIS (FIG. 3C, depicted in color and greyscale) and transwell 40-kDa FITC-dextran permeability (FIG. 3D). Our data shows that CLDN5 is a functional reporter of endothelial cell barrier and, surprisingly, it can be used to profile chemical library to find compounds that induce high endothelial barrier integrity or prevent loss of barrier breakdown. The functional reporter reacts to the in vivo permeability factor VEGFA, treatment with VEGFA which induces barrier breakdown in vivo leads to lower expression of the reporter GFP, and as such can be used as a surrogate marker for tissue barrier integrity.

### Example 4

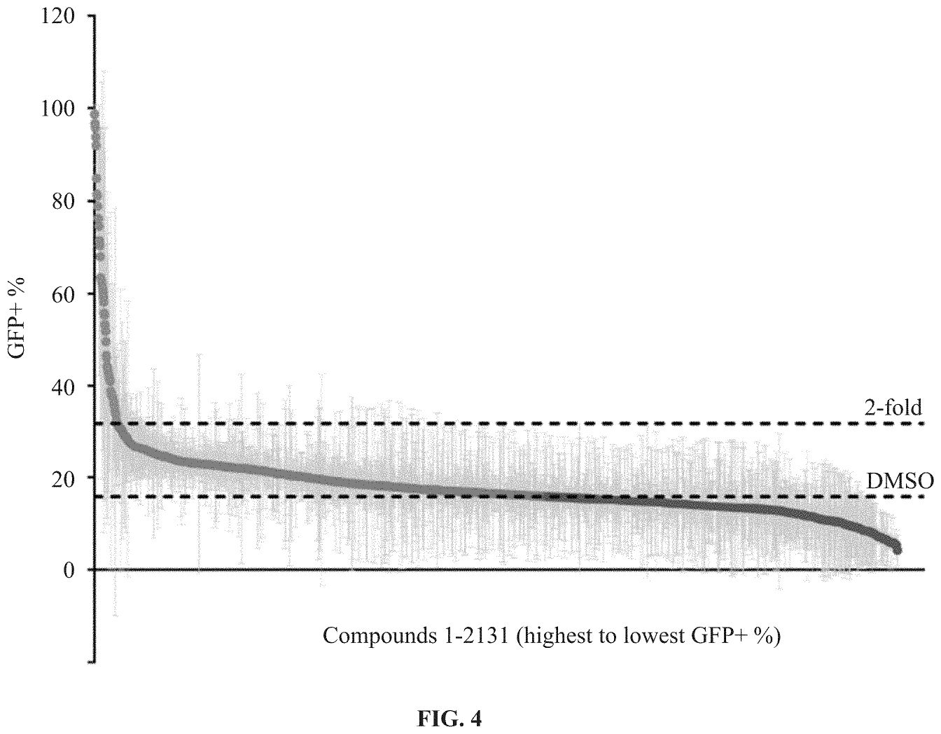

Identification of compounds inducing transendothelial barrier integrity. hPSC-EC carrying the CLDN5 reporter were screened with a drug candidate compound library and 2 days after treatment FACS measurement was performed to identify compounds that induce the percentage of GFP+ cells (FIG. 4). The focused was on compound classes that increased the % of GFP+ cells at least twofold compared to DMSO (>31.7% GFP+). Next, induction of the percentage of GFP+ cells was confirmed by performing dose-response treatment with selected potent compounds and barrier promoting activity was observed in ECIS and FITC-dextran permeability assays (data not shown). Tendency of LY215729 (TGFBR inhibitor) to promote barrier activity of resting ECs was observed which partially prevented disruption of endothelial cell layer by VEGFA. The TGFß pathway was observed to be downregulated in GFP+ cells.

### Example 5

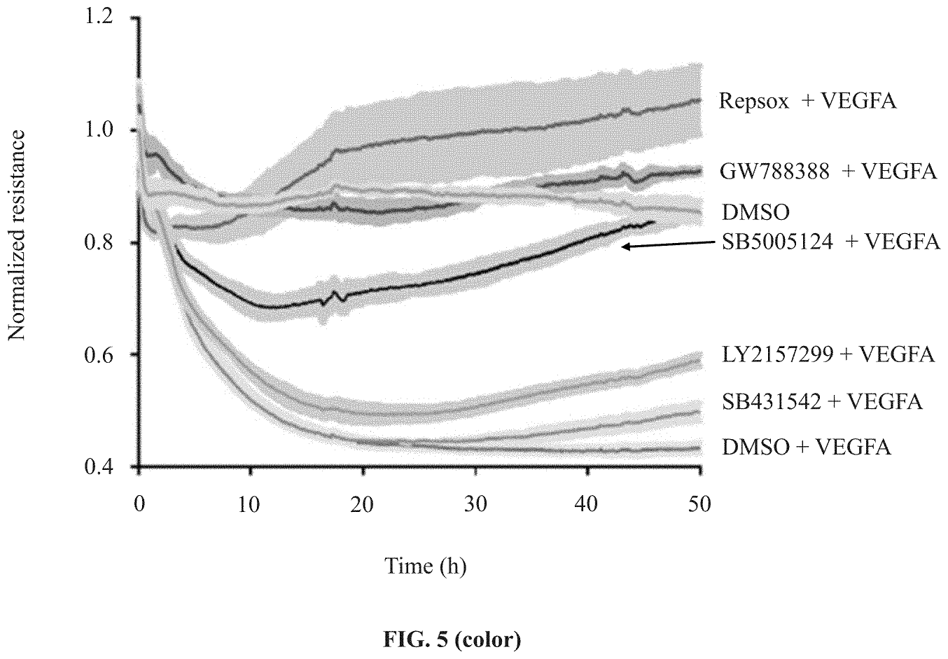

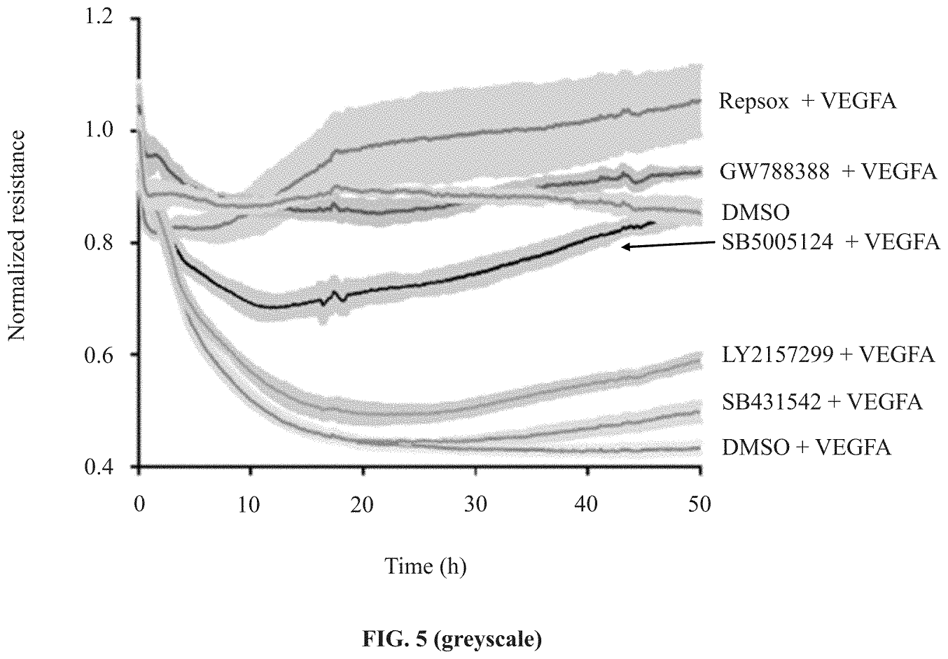

TGFBR inhibition induces transendothelial barrier integrity. In the functional barrier assays the effect on TGFR beta inhibiting compounds on EC barrier in co-application with VEGFA was assessed (FIG. 5). Under both conditions a strong EC barrier promoting effect was observed of Repsox, then GW78388 that had prevented barrier disruption with VEGFA, SB505124 had partial effect and SB431542 had no effect. Next, the specificity of several kinase inhibitors that target TGFBR were compared using a large kinase panel. All the compounds had inhibitory activity against ACVR1B and TGFBR1 but only the two most potent compounds (Repsox and GW788388) had strong inhibitory activity on TGFBR2 and weaker inhibition activity on BMPR1B (data not shown). Next, Kd for the same compounds was measured and Kd in nanomolar range was identified for all the compounds on ACVR1B and TGFBR1, but only Repsox and GW788388 had nanomolar inhibitory activities for TGFBR2 (data not shown). To elucidate the molecular mechanism behind Repsox potent induction of barrier properties RNA-seq was performed after 8 h and 48 h after treatment with TGFBR inhibitors. GSEA pathway was assessed for the most active and inactive compound analysis using the Hallmarks MsigDB database. Downregulation of TGF-beta pathway was identified for both compounds, but also differential regulation of pathways. Notably, strong upregulation of CLDN5 and downregulation of PLVAP by Repsox was observed, while expression of KDR (VEGFR2) and PECAM1 (CD31) did not change. PLVAP is shown to be suppressed in the developing blood brain barrier ECs (Hallmann R, Mayer D N, Berg E L, Broermann R, Butcher E C. Developmental dynamics. 1995; 202(4):325-32) and presence of PLVAP on ECs of BRB correlates with increased vascular permeability (Wisniewska-Kruk J, van der Wijk A E, van Veen H A, Gorgels T G, Vogels I M, Versteeg D, et al. The American journal of pathology. 2016; 186(4):1044-54.). Upregulation by Repsox of several other tight junctions or regulators of tight junctions (MARVELD3, GJA4, GJA5, IFITM3) was observed. Downregulation of RHOB which has been shown to promote barrier properties was observed. Furthermore, upregulation of Wnt target genes (AXIN2, APCDD1, TNFRSF19) and receptors for Wnt FZD4 and LRP1 was observed and downregulation of GSK3ß by Repsox. Also Repsox was the most potent compound in downregulating angiogenesis related genes (ESM1, ANGPTL4 and PPARGC1A and upregulated VEGFR1 (FLT1) that downregulates VEGFA pathway (data not shown). Repsox could downregulate several inflammation genes (NFATC2, JAK1, JAK3 and ICAM1). All tested compounds were able to downregulate TGFß pathway, Repsox being the most potent compound also inducing the SMAD6 (TGFß antagonist). Repsox also potently inhibited BMP signaling (downregulation of ENG, LRG1 and BMPR2). Most striking upregulation after RepSox treatment was of antagonists of BMP signaling (BMPER, GREM2 and GDF6). All of the antagonists of BMP signaling were involved in endothelial cell barrier stability. BMPER haplo-insufficieny has been shown to lead to increase retinal vascularization (Moreno-Miralles I, Ren R, Moser M, Hartnett M E, Patterson C. Arteriosclerosis, thrombosis, and vascular biology. 2011; 31(10):2216-22.) and to proinflammatory phenotype (Helbing T, Rothweiler R, Ketterer E, Goetz L, Heinke J, Grundmann S, et al. Blood. 2011; 118(18):5040-9). Previous reports have showed involvement of BMP signaling in inducing permeability of endothelial cells (Benn A, Bredow C, Casanova I, Vukicevic S, Knaus P. Journal of cell science. 2016; 129(1):206-18.). We have used compounds that block ALK 1, 2, 3 but we could not observe any effect on endothelial cell barrier. Also, no induction in GFP+ was observed (data not shown). In conclusion this work has identified compounds that can induce endothelial barrier resistance. In particular RepSox was identified to be able to induce strongly barrier resistance. Repsox was found in a screen that searched for compounds that replaced transgenic factor Sox2 (Ichida J K, Blanchard J, Lam K, Son E Y, Chung J E, Egli D, et al. Cell stem cell. 2009; 5(5):491-503). Induction of SOX17 and KLF4 was identified upon Repsox treatment. SOX17 (Zhou Y, Williams J, Smallwood P M, Nathans J. PloS one. 2015; 10(12):e0143650) and KLF4 (Cowan C E, Kohler E E, Dugan T A, Mirza M K, Malik A B, Wary K K. Circulation research. 2010; 107(8):959-66) has been shown to promote barrier formation previously. In conclusion, a transcriptional reporter for CLDN5 was generated and used to screen a library of chemical compounds that covers a large number of drug candidates to find inhibitors of TGFß that prevent disruption of endothelial cell barrier by VEGFA and in particularly Repsox that can potently induce transendothelial barrier integrity, and, therefore is selected for further analysis on tissue barrier integrity in vivo.

1-25. (canceled)

26. An in vitro method, comprising the steps of:
a) providing endothelial cells (ECs) comprising a reporter gene under the control of a tight junction gene promoter, wherein the ECs are enriched for cells expressing the reporter gene;
b) contacting the ECs with a drug candidate; and
c) measuring in vitro transendothelial barrier integrity (TBI) before and after contacting the ECs with the drug candidate, or measuring in vitro TBI of the ECs contacted with the drug candidate and in parallel measuring in vitro TBI of ECs not contacted with the drug candidate;
wherein the method identifies a drug candidate capable of i) increasing in vivo transendothelial barrier integrity (TBI) or ii) decreasing in vivo TBI of endothelial cells (ECs); wherein:
(i) a higher in vitro TBI of the ECs contacted with the drug candidate compared with the in vitro TBI of the ECs not contacted with the drug candidate is indicative of a drug capable of increasing in vivo TBI of ECs, and
(ii) a lower in vitro TBI of the ECs contacted with the drug candidate compared with the in vitro TBI of the ECs not contacted with the drug candidate is indicative of a drug capable of decreasing in vivo TBI of ECs.

27. The method of claim 26, wherein step c) comprises measuring the transendothelial electrical resistance (TEER) wherein the measured TEER is indicative for in vitro TBI.

28. The method of claim 26, wherein step c) comprises measuring the expression of the reporter gene wherein the expression of the reporter gene is indicative for in vitro TBI.

29. The method of claim 26, wherein the tight junction gene is selected from the group consisting of CLDN5, ocludin (OCLN) and MARVELD3.

30. The method of claim 26, wherein the ECs are differentiated from pluripotent stem cells.

31. The method of claim 26, wherein a polynucleotide encoding the reporter gene is inserted at the 3′ end of the tight junction gene.

32. The method of claim 31, wherein (i) a tight junction gene reporter gene fusion protein is expressed, or (ii) the reporter gene is expressed from an internal ribosomal entry site (IRES), or (iii) a tight junction gene reporter gene fusion protein is expressed and subsequently processed to individual tight junction protein and reporter protein.

33. The method of claim 32, wherein (iii) a tight junction gene reporter gene fusion protein is expressed and subsequently processed to individual tight junction protein and reporter protein, wherein a polynucleotide encoding a self-cleaving peptide is introduced between the tight junction gene and the reporter gene.

34. The method of claim 26, wherein activation of the promoter of the tight junction gene leads to expression of the reporter gene.

35. The method claim 26, wherein the cells are enriched for cells expressing the reporter gene in step a) by fluorescence activated cell sorting (FACS) or magnetic activated cell sorting (MACS).

36. The method of claim 26, which is performed in a high-throughput format.

37. The method of claim 26, which is used to screen molecules in a drug development setting, in particular for high-throughput screening a drug candidate compound library.

38. A cell culture produced according to step a) of claim 26, wherein the fraction of cells expressing the tight junction gene is higher than 30%, 40%, 50%, 60%, 70%, 80%, 90% or 95%.

39. A cell capable of expressing a reporter gene, wherein expression of the reporter gene is under the control of the promoter of a tight junction gene.

40. The cell of claim 39, wherein the tight junction gene is CLDN5.

41. A method of treating a disease in an individual, comprising:
administering to said individual a therapeutically effective amount of a composition comprising 2-[3-(6-Methyl-2-pyridinyl)-1H-pyrazol-4-yl]-1,5-naphthyridine in a pharmaceutically acceptable form; or
administering to said individual a therapeutically effective amount of a composition comprising 4-[4-[3-(2-Pyridinyl)-1H-pyrazol-4-yl]-2-pyridinyl]-N-(tetrahydro-2H-pyran-4-yl)-benzamide in a pharmaceutically acceptable form.

42. The method of claim 41, wherein said disease is associated with vascular complications.

43. The method of claim 41, wherein said disease is selected from the group consisting of diabetes Type-2 and Type-1, diabetic retinopathy, Wet AMD, Metabolic Syndrome, Severe Obesity, Hypercholesterolemia, Hypertension, coronary artery disease, nephropathy, retinopathy, kidney failure, tissue ischemia, chronic hypoxia, artherosclerosis, and tissue edema caused by drug-induced toxicity.

44. The method of claim 41, wherein said disease is diabetic retinopathy or Wet AMD.
